(19) United States
(12) Patent Application Publication
(10) Pub. No.: US 2010/0234323 A1
(43) Pub. Date: Sep. 16, 2010

(54) Antimicrobial Acids and Salts

(76) Inventors: Werner Hölzl, Eschentzwiller (FR); Andrea Preuss, Basel (CH)

Correspondence Address: BASF Performance Products LLC;Patent Department, 540 White Plains Road, P.O. Box 2005, Tarrytown, NY, 10591, US

(21) Appl. No.: 12/227,674

(22) PCT Filed: Jun. 4, 2007
(86) PCT No.: PCT/EP2007/055466

§ 371 Date: Nov. 24, 2008

(30) Foreign Application Priority Data
Jun. 2, 2006 (EP) 06114888.8

(51) Int. Cl. A61K 31/695; C07F 9/02

(52) U.S. Cl. 514/63; 556/405; 556/437

(57) ABSTRACT

The invention relates to novel acidic siloxane derivatives, the use, methods of use or processes making use of siloxane derivatives of this type, especially to achieve an anti-microbial, preservative and/or antiadhesive effect, for the protection of articles and/or materials, and a process for the manufacture of the novel compounds. The siloxane acids (which may also be in salt form) have the formula (I), formula (II) and/or formula (III), wherein the symbols have the meanings given in the specification.

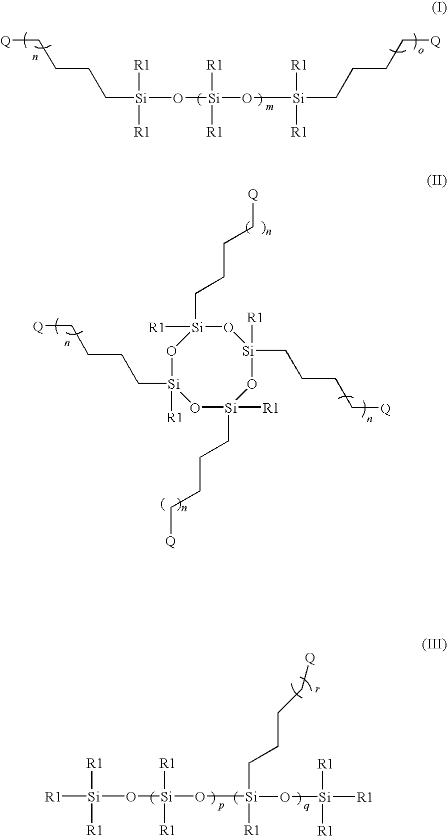

### SUMMARY OF THE INVENTION

The invention relates to the use (or a method of use or process making use) of certain siloxane acid compounds as antimicrobial, preservative and/or microorganism adhesion inhibitors for the protection of articles and/or materials, as well as novel siloxane acid compounds, processes for their manufacture and compositions comprising them.

### BACKGROUND OF THE INVENTION

In order to avoid or inhibit growth of microorganisms on or in materials or objects, a variety of methods have been used. For example, it has been tried to achieve such results by incorporating nano-particles, e.g. on the basis of silver or TiO₂: For example, the incorporation of nanoparticles into organic resins or ceramics has been proposed. Alternatively, the deposition in plasma vacuum with deposition of nanoparticles and the like into coatings have been suggested. Furthermore, certain surfactants are in principle known to be useful as means for cleaning and sometimes even disinfecting surfaces of goods, such as industrial products or consumer articles.

Colonization by microbes on a wide variety of surfaces and in a variety of materials can cause phenomena such as dirty appearance, smell and even serious hygienic and health problems. Thus there is considerable interest in the development of hygienic materials and surfaces which provide biocidal activity and are, at least to some extent, easy to clean or even self-cleaning.

JP 2001-226486 and US 2003/0211051 A1 mention certain carboxyl-group carrying organosiloxanes which, in the Japanese application, can be used in the raw material of a drainage system coating, in the US-application for administration in the care of teeth and other surfaces of the oral cavity.

What is required, though, also in view of the ability of microorganisms to adapt to a variety of adverse circumstances including the development of resistance, are novel surface, material and/or product derivatization methods and materials, e.g. classes of compounds that allow to inhibit microbial growth or even show microbicidal activity against a broad range of microorganisms, e.g. one or more microorganisms selected from bacteria, fungi, yeasts and algae, and/or even have negative influence on the settlement of multicellular organisms, such as algae, mosses or ferns, on and/or in materials or objects and thus are useful inter alia for applications in preservation, as additives in plastics, in coatings, on textiles, in paper, in cosmetics, in pharmaceutical formulations or corresponding containers, in home or personal care applications and the like, be it with natural and/or with synthetic materials, and for other corresponding uses.

### GENERAL DESCRIPTION OF THE INVENTION

It has been found surprisingly now that a class of compounds shows advantageous properties in this regard which belong to a class of siloxanes substituted with acidic groups, as these compounds show a technical or functional feature of being able to achieve an antimicrobial, preservative and/or microorganism adhesion inhibiting effect for the protection of said article and/or material, especially (direct) antimicrobial effects.

### DETAILED DESCRIPTION OF THE INVENTION

The present invention relates to the a process or method of achieving an antimicrobial, preservative and/or microorganism adhesion inhibiting effect for the protection within or on the surface of an article and/or material comprising applying an acid compound (as such, as an acid compound mixture or in the form of a composition) comprising,

an acid of the formula I,

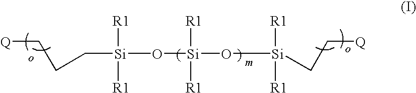

wherein R1=lower alkyl, especially methyl;
Q represents a linear or branched C₁-C₁₀-alkyl moiety substituted with 1-3 functional groups independently selected from the group consisting of COOH, P(O)(OH)₂ and S(O)₂(OH) which are attached via a C-atom to said moiety;
m represents an integer from 1 to about 20;
o represents an integer from 0 to 15; and
   the acid preferably has a polydispersity of up to about 3 and a weight-average molecular weight of 600 to about 3000;
   an acid of the general formula II,

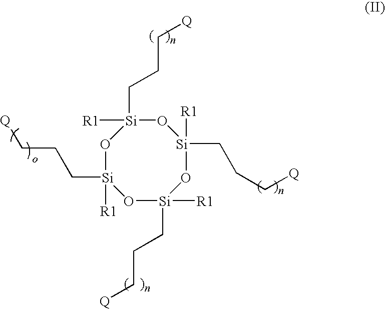

wherein
R1=methyl
Q represents a linear or branched C₁-C₁₀-alkyl moiety substituted with 1-3 functional groups independently selected from the group consisting of COOH, P(O)(OH)₂ and S(O)₂(OH) which are attached via a C-atom to said moiety; and
n represents an integer from 0 to 15;
   and/or
   an acid of the formula III,

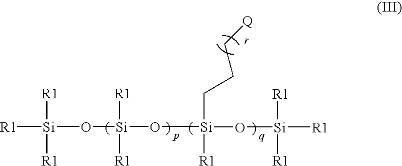

wherein:
R1=methyl
Q represents a linear or branched C₁-C₁₀-alkyl moiety substituted with 1-3 functional groups independently selected from the group consisting of COOH, P(O)(OH)₂ and S(O)₂(OH) which are attached via a C-atom to said moiety;
p represents an integer from 1 to about 20;
q represents an integer from 1 to about 20;
r represents an integer from 0 to 15; and
   the acid preferably has a polydispersity of up to about 3 and a weight-average molecular weight of 600 to about 3000;
   especially with the proviso that, if the acid compound used is one comprising only one or more acids of the formula III wherein Q is selected solely from the group consisting of linear or branched C₁-C₁₀-alkyl moieties substituted with two functional COOH groups attached via a C-atom to said moiety,
   either the use is limited to a use in order to achieve an antimicrobial and/or a preservative effect, or, where the use is in order to provide a microorganism adhesion inhibiting effect, the use is limited to coating or impregnation of materials comprising (especially consisting of) synthetic polymers or polymer precursors or monomeric compounds, or of articles made from such materials, or to the bulk addition to a material or an article; or (for these or the other uses comprised above) in addition to one or more acids of the formula III at least another acid compound comprising an acid of the formula I as defined above, an acid of the formula II as defined above; and/or at least one acid compound comprising an additional different acid of the formula III as mentioned above wherein at least one moiety Q is present which is a linear or branched C₁-C₁₀-alkyl moiety substituted with 1 to 3 COOH, P(O)(OH)₂ and/or S(O)₂(OH) groups, with the proviso that in the case of a compound of the formula III said moiety Q does not carry only 2 COOH⁻ groups is applied in addition;
   where the acid(s) of the formulae I, II and/or III may also partially or completely be present as salts (where in an acid of the formula I, II and/or III a part or all of the mentioned COOH, P(O)(OH)₂ and/or S(O)₂(OH) groups may also be present (e.g. in an acid base equilibrium) in negatively charged (partially or about completely de-protonated) form with a cation as counterion leading at least substantially to charge neutralization);
   as well as the use of said acid compound(s) or salt(s) thereof, mixtures comprising them and/or compositions comprising them with the provisos given above, comprising applying at least one of these compounds and/or salt(s) thereof, mixtures thereof or compositions comprising them, to an article and/or material in order (=with the purpose or the functional feature) to achieve an antimicrobial, preservative and/or microorganism adhesion inhibiting effect for the protection of said article and/or material.

The invention also relates to novel acid compounds, compound mixtures or compositions comprising

at least one acid comprising at least one moiety Q which represents a linear or branched C₁-C₁₀-alkyl moiety substituted with 1-3 functional groups independently selected from the group consisting of COOH, P(O)(OH)₂ and S(O)₂(OH) which are attached via a C-atom to said moiety, wherein said at least one acid is selected from one or more acids from the group consisting of
one or more acids of the formula I,

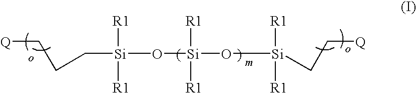

wherein R1=lower alkyl, especially methyl;
Q is as just defined;
m represents an integer from 1 to about 20;
o represents an integer from 0 to 15; and
   the acid preferably has a polydispersity of up to about 3 and a weight-average molecular weight of 600 to about 3000;
   one or more acids of the general formula II,

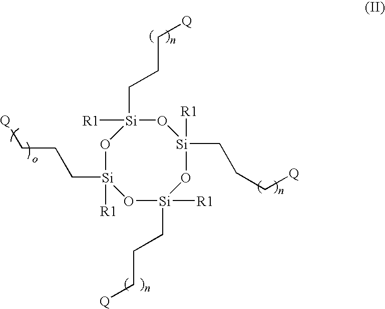

wherein
R1=methyl
Q is as just defined; and
n represents an integer from 0 to about 15;
   and/or
   one or more acids of the formula III,

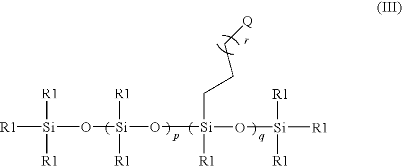

wherein:
R1=methyl
Q is as just defined;
p represents an integer from 1 to about 20;
q represents an integer from 1 to about 20;
r represents an integer from 0 to 15; and
   the acid preferably has a polydispersity of up to about 3 and a weight-average molecular weight of 600 to about 3000;
   with the proviso that if only (one or more) acids of the formula III are present in a given compound, compound mixture or composition, at least in a part of the molecules of the formula III one of the moieties Q is a linear or branched C₁-C₁₀-alkyl moiety substituted with 1 to 3 functional groups selected from P(O)(OH)₂ and S(O)₂(OH) or with 1 or 3 COOH groups;
   and/or (a) salt(s) thereof.

The invention further relates to compositions comprising one or more of the acid compounds or acids (these two expressions are used herein synonymously) mentioned in the preceding paragraph (also sometimes termed “compositions of the invention” hereinafter, implying also the use of such compositions according to the invention) which are appropriate especially for application to materials and articles, e.g. for covering materials or articles, for addition to materials or articles, e.g. by being admixed to materials during their manufacture, and/or for impregnating materials or articles, where said compositions may comprise an acid compound of the invention and may in addition comprise other additives or carrier materials, such as binders, solvents, buffers or the like.

Among the compositions of interest, there may especially be mentioned, antimicrobial compositions, antifouling compositions, coating compositions or materials (coatings), coating systems, cosmetic formulations (including oral preparations), home care compositions, pharmaceutical compositions (including oral preparations), antimicrobial preparations, laundry detergent and/or fabric care compositions, each independently forming a preferred embodiment and preferably defined as below. Especially compositions with the novel acid compounds of the invention are of interest.

In a further embodiment, the invention relates to the use of one or more acid compounds or compositions according to the invention as defined above or preferably below in order to achieve an antimicrobial, preservative and/or microorganism adhesion inhibiting effect for the protection of one or more articles and/or materials, said use especially comprising adding one or more of said acid compounds or a composition comprising one or more of said acid compounds to said article(s) and/or material(s), especially for use as antimicrobial. The addition may be by integration into the material (e.g. by admixing during manufacture of a product, such as an article or a material), by impregnation of an article or material and/or by application to a surface e.g. of an article or material.

Yet a further embodiment of the invention relates to a process of manufacture of one or more of the novel acid compounds of the invention as defined above or below, and/or to a process of manufacture of a novel composition comprising one or more acids according to the invention.

The acids according to the invention can be prepared by methods analogous to methods that per se are known in the prior art, though not for the present novel acid compounds so that the processes with regard to these are novel and part of the invention, namely by
(a) for the manufacture of an acid of the formula I, and/or a salt thereof, reacting a compound of the formula IA,

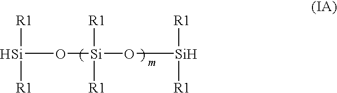

wherein R1 and m are as defined for an acid of the formula I,
under hydrosilylation conditions with a vinyl compound of the formula IV,

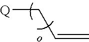

wherein Q and o are as defined for an acid of the formula I, or
(b) for the manufacture of an acid of the formula II, and/or a salt thereof, reacting a compound of the formula IIA,

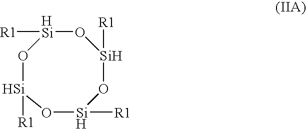

wherein R1 is as defined for an acid of the formula II,
under hydrosilylation conditions with a vinyl compound of the formula IV*,

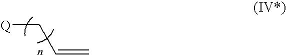

wherein Q and n are as defined for an acid of the formula II, or
(c) for the manufacture of an acid of the formula III, reacting a compound of the formula IIIA,

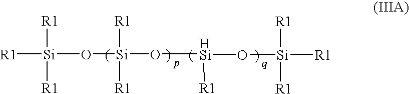

wherein R1, p and q are as defined for an acid of the formula III,
under hydrosilylation conditions with a vinyl compound of the formula IV**,

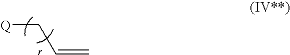

wherein Q and r as defined for an acid of the formula III,
with the proviso that in any of the starting materials of the formula IV, IV* and IV**, functional groups COOH, P(O)(OH)₂ and/or S(O)₂(OH) are present in protected form;
or leading two or more of the reactions under (a), (b) and (c) and the subsequent removal of protecting groups in parallel in order to directly obtain mixtures of acid compounds with acids of the formula I, II and/or III;
if functional groups are present (as can be the case if no parallel deprotection takes place) in protected form, subsequently removing the protection from the functional groups;
and, if desired, a free acid obtainable according to a process according to a), b) and/or c) and deprotection is converted into a salt, or a salt of an acid compound obtainable according to a process according to a), b) and/or c) is converted into a different salt with a different cation.

The hydrosilylation according to any one of the processes (a), (b) or (c), or any combination thereof, is preferably carried out in an inert organic solvent or solvent mixture, such as toluene, preferably at elevated temperature, e.g. from 50° C. to the reflux temperature of the reaction mixture, e.g. at about 90° C., in the presence of catalytic amounts of a transition metal complex, such as complexes from rhodium, iridium, cobalt or more specifically carbonyl compounds derived from iron, cobalt, nickel, rhodium, ruthenium, manganese and/or chromium, most preferably complexes derived from platinum or ehxachloroplatinic acid (H₂PtCl₆), such as especially Karstedt-catalyst (di-platinum-tris(divinyltetramethyldisiloxan)) catalyst. Preferably, the stoichiometry is chosen such that all silicium bound hydrogen atoms in the compounds of the formulae IA, IIA and IIIA are reacted with a compound of the formula IV, IV* and IV**, respectively.

The protected forms of functional groups COOH, P(O)(OH)₂ and/or S(O)₂(OH) are preferably esters, e.g. with lower alkyl alcohols, or anhydrides, e.g. formed between adjacent COOH, P(O)(OH)₂ and/or S(O)₂(OH) groups or as mixed anhydrides with further acids, e.g. acetic acid. The removal of the protection preferably takes place by hydrolysis (especially in the case of carbonic esters), e.g. in the presence of an aqueous solvent, such as toluene and water, at elevated temperatures, e.g. at temperatures from 40° C. to the reflux temperature of the reaction mixture; or (especially in the case of phosphonic acid esters) in an appropriate solvent, such as dry acetonitrile, in the presence of a halosilane, such as bromotrimethylsilane, at temperatures e.g. from 0 to 50° C., followed by treatment with methanol e.g. at elevated temperatures, for example from 50° C. to reflux temperature of the reaction mixture.

The introduction of protection follows standard procedures known in the art, e.g. as mentioned in T. W. Greene and P. G. Wuts, “Protective Groups in Organic Synthesis”, 3^{rd} edition, John Wiley & Sons, Inc., New York 1999.

Acid compounds of the invention can be converted into salts or salts thereof into different salts by adding or replacing, respectively, cations thereto/therein by (other) cations according to customary methods, e.g. by precipitation in the presence of metal or ammonium salts with the desired cations or using cation exchangers.

The preparation of compositions according to the invention comprises admixing one or more acid compounds, or salts thereof, with one or more other additives, e.g. those mentioned below.

The general terms used hereinbefore and hereinafter preferably have within the context of this disclosure the following meanings/definitions, unless otherwise indicated:

“Lower” preferably means that the corresponding moiety has up to and including 7, more preferably up to and including 4 carbon atoms, and may be linear or branched one or more times. For example, lower alkyl preferably stands for methyl, ethyl, n-propyl, isopropyl, n-butyl, iso-butyl, sec-butyl, tert-butyl, pentyl, hexyl or heptyl.

A linear or branched C₁-C₁₀-alkyl moiety can be linear of branched one or more times. Such a moiety Q wherein 1-3 functional groups independently selected from the group consisting of COOH, P(O)(OH)₂ and/or S(O)₂(OH) are attached via a C-atom to said moiety preferably carries 3 or more preferably two of these functional groups, more preferably only identical groups. Preferably the functional groups are selected from the group consisting of P(O)(OH)₂ and/or S(O)₂(OH) which lead to especially good antimicrobial activity

Acid compounds, acid compound mixtures or compositions comprising such acid compound(s) or mixtures (especially those described as being preferred) are also referred to as “antimicrobial agents” hereinafter.

Where in the present disclosure an acid (compound) comprising or consisting of an acid according to the invention or an acid (=type of acid) of the formula I, II and/or III is mentioned, this is intended to include the case where more than one such acid (=type of acids) is present falling under the definition of the respective “acid” of formula I, II and/or III—in fact, often (due to the corresponding starting materials) mixtures of more than one such acid (e.g. with different number of repetition units in formula I or III or different number of substituents Q) are present, so that in fact usually “an acid (compound) of the formula . . . ” can in fact be a mixture of acids that fall under the corresponding formula, thus meaning that “ana acid (compound)” is in fact a mixture of acid (compound)s. Therefore, “an acid (compound)” also can include more than one such acid (compound), that is, a compound mixture falling under the corresponding definition. The same is also true for precursors and starting materials and in the Examples. Where one or more acids are mentioned, this can refer to one type or more than one type of acids (e.g. mixtures of acids or one pure type of acids). Where the plural (“Acids, “acid compounds”, “cations”, “precursors”, starting materials” or the like) form is used, this also includes one acid, acid compound, cation, precursor, starting material or the like, respectively, and vice versa, where not excluded specifically or not possible according to the knowledge of a person skilled in the art.

Where acids or acid compounds of the formula I, II and/or III are mentioned, this includes salts with cations thereof and/or (e.g. equilibrium) mixtures of such free acids and salts thereof, where at least a pat of the COOH, P(O)(OH)₂ and/or S(O)₂(OH) groups is present in deprotonated form.

Where salts of any one or more of the acid (compound)s of the formula I, II and/or III are mentioned, these comprise within Q partially or totally deprotonated COOH, P(═O)(OH)₂ and/or S(O)₂(OH) groups, that is COO [symbol] , P(O)(OH)(O [symbol] ), P(O)(O [symbol] )₂ and/or S(O)₂(O [symbol] ), and in addition one or more cations to substantially neutralize the corresponding salt(s).

A cation or cations (counterion(s) leading to charge neutralisation) are present to form a corresponding substantially neutral salt of an acid of any one or more of the formulae I, II and or III—this means preferably that in an acid according to the invention or in a salt used in accordance with the invention per acid of the formula I, II and/or III in deprotonated form, a number of cations is present that substantially leads to charge neutralization.

However, e.g. on surfaces it may also be possible that counterions are stripped away so that a net charge may result at least temporarily—this is for example intended to be meant by the term “substantially leads to charge neutralization”.

As examples for possible cations, the following can be mentioned: Non-substituted or substituted linear or circular ammonium cations, e.g. mono- to tetra(e.g. independently chosen lower alkyl and/or phenyl-lower alkyl)-substituted ammonium, corresponding diammonium cations, dimethylpiperidinium, alkanolammonium (e.g. mono-, di- or triethanolammonium), or preferably metal cations, such as alkali metal cations, preferably sodium, potassium or lithium cations, or earth alkaline metal salts, such as calcium or barium cations. Also other cations are possible, e.g. with more than one positively charged group or with positively charged and negatively charged groups with an excess of positive charges over the negative charges. Also other charged organic or inorganic cations are possible, such as positively charged dye cations or positively charged metal complex ions.

Where the term “comprising” is used, this is intended to mean that the component, components, feature or features mentioned or enumerated thereafter may be fulfilled not only alone, but that also one or more other components and/or features (e.g. other additives, other actions) may be present in addition to those specifically mentioned. This is in contrast to the term “containing” or “consisting of” which mean that no other components or features are included except for those specifically mentioned after such an expression and thus denote a complete enumeration/representation of features and/or components. Wherever “comprising” is used, this may (independently of other occurrences) be replaced by the narrower term “consisting of” or (in case of processes or methods) by “containing the step of”, where possible and expedient, thus leading to specific and preferred embodiments of the invention.

A preferred molecular weight of an acid of the formula I or the formula III is in the range from about 600 to about 3000.

A preferred polydispersity (ratio of the weight average to the number average molecular weight) of said acids is in the range of up to about 3.

“About” wherever used in the present disclosure means that a certain deviation from a numerical value may be present and the corresponding value is not intended to mean an absolute boarder as will be apparent to a person skilled in the art; it preferably means “±20%” of the respective numerical value, more preferably “±10%”, yet more preferably “±5%” thereof, and most preferably can be deleted so that only the respective numerical value remains without preceding “about”.

Antimicrobial activity means an at least partially microorganism inactivating (viability affecting), especially antibiotic or microbicidal (microorganism impeding or especially killing) activity that leads to a direct partial or complete inhibiting effect on microorganisms, such as especially bacterial, protozoic, fungal and/or algal cells or multicellular microorganisms, especially antibacterial or antifungal properties, especially against those microorganisms mentioned in the Examples, or in a broader sense also to inhibiting effects on virus or phages. The effect is especially on the basis of a negative effect on the metabolism, structure or reproduction (e.g. cell division or steps preceding it) of the microorganism(s), e.g. a toxic effect.

Preservative activity means especially that articles (goods in any form) and/or materials are preserved against a decomposing activity, especially by decomposition of one or more of its structural components due to microorganism attack on structural components of articles or materials. For example, in the case of pharmaceutical compositions or food preservative activity especially means that the structure of an active chemical entity or important food constituents such as vitamins are preserved against decomposition by microorganisms.

Microorganism adhesion inhibiting activity means especially that the colonization of an article or material by microbes is diminished or completely abolished due to mainly purely structural effects such as the provision of a surface structure that impedes the binding of microorganisms or other materials that allow for the anchoring of microorganisms. Thus the basis is not an effect on the viability of the microorganism but an effect on the ability of the microorganism to physically settle on or in a material or article.

Apply, application of, addition of or add(ing) and the like especially means to coat, impregnate or mix with.

Bulk addition especially refers to the addition to a material by admixing an acid compound, acid compound mixture or a composition comprising an acid compound or acid compound mixture comprising one or more acids of the formula I, II and/or III to the material of the article or material, during its manufacture (e.g. by admixing to starting materials, e.g. granules, powders, solutions or the like), in contrast to coating or impregnation which correspond to application on an already manufactured material or article. It also comprises addition to powders such as materials for pharmaceuticals.

In principle, the acids etc. of the formula I, II and/or III may be applied by integration, admixing, impregnation, impregnating and/or coating includes homogenous integration or admixing, inhomogenous integration or admixing, complete or partial impregnation and/or complete or partial coating.

Compositions according to the invention, which can also be called antimicrobial compositions hereinafter, may, in addition to an acid compound of the formula I, II and/or III, which may also be present as sole component, comprise one or more other additives such as a binder, solvents and the like. The invention also comprises the use of an acid compound according to the invention or such a composition, especially a novel acid compound, a mixture of such acid compounds (especially one comprising one or more novel acid compounds) or a composition comprising these.

The present invention also relates to antimicrobial compositions or their use to achieve anti-microbial (especially preferred), preservation and/or microorganism adhesion inhibiting effects, with the provisos mentioned above, comprising an organic carrier material (component (A)) and an acid compound or acid compound compound mixture according to the invention (component (B)).

Preferred organic carrier materials are polymers, like those given below, in particular synthetic polymers, for example thermoplastic polymers. Polyamides, polyurethanes and polyolefins are particularly preferred. Examples of preferred polyolefins are polypropylene or polyethylene.

Of special interest are also compositions wherein the composition is a coating composition and component (a) is an organic film-forming binder.

Of special interest are transparent coating compositions which after curing lead to transparent coatings.

The composition, e.g. coating, may be solvent borne or aqueous. Aqueous compositions are typically considered more environmentally friendly. The coating or other composition according to the invention is, for example, aqueous dispersion of an acid compound according to the invention and a binder or a water based coating or paint. The coating composition is preferably a coating material or paint, especially an aqueous coating material or an aqueous paint.

The antimicrobial compositions of present invention are for example used as a coating applied to a surface which is exposed to conditions favorable for bioaccumulation.

The antimicrobial composition of the present invention may be part of a complete coating or paint formulation, such as a marine gel-coat, shellac, varnish, lacquer or paint, or the anti microbial composition may comprise only an acid compound (or mixture) of the invention and binder, or an acid compound (or mixture) of the invention, binder and one or more additives. It is anticipated that other additives encountered in such coating formulations or applications will find optional use in the present applications as well.

Examples of coating materials are lacquers, paints or varnishes. These always contain an organic film-forming binder in addition to other, optional components.

Preferred organic film-forming binders are epoxy resins, polyurethane resins, amino resins, acrylic resins, acrylic copolymer resins, polyvinyl resins, phenolic resins, styrene/butadiene copolymer resins, vinyl/acrylic copolymer resins, polyester resins, UV-curable resins or alkyd resins, or a mixture of two or more of these resins, or an aqueous basic or acidic dispersion of these resins or mixtures of these resins, or an aqueous emulsion of these resins or mixtures of these resins.

Biocompatible coating polymers, such as poly[alkoxyalkanoate-co-3-hydroxyalkenoate (PHAE) polyesters (see e.g. Geiger et al., Polymer Bulletin 52, 65-70 (2004), can also serve as binders in the present invention.

Of particular interest are organic film-forming binders for aqueous coating compositions, e.g. alkyd resins; and hybrid systems based on, for example, epoxy acrylates.

More specifically, the alkyd resins can be water-dilutable alkyd resin systems which can be employed in air-drying form or in the form of stoving systems, optionally in combination with water-dilutable melamine resins; the systems may also be oxidatively drying, air-drying or stoving systems which are optionally employed in combination with aqueous dispersions based on acrylic resins or copolymers thereof, with vinyl acetates, etc.

The acrylic resins can be pure acrylic resins, epoxy acrylate hybrid systems, acrylic acid or acrylic ester copolymers, combinations with vinyl resins, or copolymers with vinyl monomers such as vinyl acetate, styrene or butadiene. These systems can be air-drying systems or stoving systems.

Preferred epoxy resins are those based on aromatic polyols, especially those based on bisphenols. The epoxy resins are employed in combination with crosslinkers. The latter may in particular be amino- or hydroxy-functional compounds, an acid, an acid anhydride or a Lewis acid.

Polyurethane resins are derived from polyethers, polyesters and polybutadienes with terminal hydroxyl groups, on the one hand, and from aliphatic or aromatic polyisocyanates on the other hand.

Preferably, the polyurethanes are prepared in situ from polyethers, polyesters and polybutadienes with terminal hydroxyl groups, on the one hand, and from aliphatic or aromatic polyisocyanates on the other hand.

Examples of suitable polyvinyl resins are polyvinylbutyral, polyvinyl acetate or copolymers thereof.

Suitable phenolic resins are synthetic resins in the course of whose construction phenols are the principal component, i.e. in particular phenol-, cresol-, xylenol- and resorcinol-formaldehyde resins, alkylphenolic resins, and condensation products of phenols with acetaldehyde, furfurol, acrolein or other aldehydes. Modified phenolic resins are also of interest.

UV-(ultraviolet) curable resins may contain one or more olefinic double bonds. They may be of low (monomeric) or relatively high (oligomeric) molecular mass. Examples of monomers containing a double bond are alkyl or hydroxyalkyl acrylates or methacrylates.

Examples of monomers containing two or more double bonds are ethylene glycol and propylene glycol di(meth)acrylates.

Examples of relatively high molecular mass (oligomeric) polyunsaturated compounds are acrylated epoxy resin and acrylated or vinyl ether- or epoxy-functional polyesters, polyurethanes and polyethers. Especially suitable are combinations of polymers and oligomers which carry vinyl ether groups, as described in WO-A-90/01512.

Also suitable are compounds containing one or more free-radically polymerizable double bonds. In these compounds the free-radically polymerizable double bonds are preferably in the form of (meth)acryloyl groups. (Meth)acryloyl and, respectively, (meth)acrylic here and below means acryloyl and/or methacryloyl, and acrylic and/or methacrylic, respectively. Preferably, at least two polymerizable double bonds are present in the molecule in the form of (meth)acryloyl groups. The compounds in question may comprise, for example, (meth)acryloyl-functional oligomeric and/or polymeric compounds of poly(meth)acrylate.

Functionalized acrylates are also suitable. Examples of suitable monomers which are normally used to form the backbone (the base polymer) of such functionalized acrylate and methacrylate polymers are acrylate, methyl acrylate, methyl methacrylate, etc. Additionally, appropriate amounts of functional monomers are copolymerized during the polymerization in order to give the functional polymers.

Particularly suitable compounds are, for example, esters of ethylenically unsaturated monofunctional or polyfunctional carboxylic acids and polyols or polyepoxides, and polymers containing ethylenically unsaturated groups in the chain or in side groups

Examples of suitable monofunctional or polyfunctional unsaturated carboxylic acids are acrylic acid, methacrylic acid

It is, however, also possible to use saturated dicarboxylic or polycarboxylic acids in a mixture with unsaturated carboxylic acids.

Suitable polyols include aromatic and especially aliphatic and cycloaliphatic polyols. Preferred Examples of aromatic polyols are hydroquinone, 4,4′-dihydroxybiphenyl, 2,2-di(4-hydroxyphenyl)propane, and also novolaks and resols.

Examples of aliphatic and cycloaliphatic polyols are alkylenediols having preferably from 2 to 12 carbon atoms, such as ethylene glycol, 1,2- or 1,3-propanediol.

The polyols may have been partly or fully esterified with one or more different unsaturated carboxylic acids, the free hydroxyl groups in partial esters possibly having been modified, e.g. etherified or esterified with other carboxylic acids.

Suitable UV-curable resins include the amides of identical or different unsaturated carboxylic acids with aromatic, cycloaliphatic and aliphatic polyamines having preferably from 2 to 6, particularly from 2 to 4 amino groups. Examples of such polyamines are ethylenediamine, 1,2- or 1,3-propylenediamine, Further suitable polyamines are polymers and copolymers containing possibly additional amino groups in the side chain, and oligoamides having amino end groups.

Suitable unsaturated polyesters and polyamides are derived, for example, from maleic acid and diols or diamines. The maleic acid may have been replaced in part by other dicarboxylic acids. They may be used together with ethylenically unsaturated comonomers, e.g. styrene. The polyesters and polyamides may also be derived from dicarboxylic acids and ethylenically unsaturated diols or diamines, especially from relatively long chain ones having, for example, from 6 to 20 carbon atoms.

Polybutadiene and polyisoprene and copolymers thereof are known. Examples of suitable comonomers are olefins such as ethylene, propene, butene, hexene, (meth)acrylates, acrylonitrile, styrene or vinyl chloride. Polymers containing (meth)acrylate groups in the side chain are likewise known. They may comprise, for example, reaction products of novolak-based epoxy resins with (meth)acrylic acid

The UV-curable resins may be used alone or in any desired mixtures. Preference is given to using mixtures of polyol (meth)acrylates.

It is also possible to add binders to the compositions of the invention, which is especially appropriate when the photopolymerizable compounds are liquid or viscous substances.

The unsaturated compounds may also be used in a mixture with non-photopolymerizable film-forming components, such as glycols, oils, waxes and/or surfactants. These may be, for example, physically drying polymers or their solutions in organic solvents, such as nitrocellulose or cellulose acetobutyrate, for example. They may also, however, be chemically and/or thermally curable resins, such as polyisocyanates, polyepoxides or melamine resins, for example. By melamine resins are meant not only condensates of melamine (1,3,5-triazine-2,4,6-triamine) but also those of melamine derivatives. In general, the components comprise a film-forming binder based on a thermoplastic or thermosettable resin, predominantly on a thermosettable resin.

Component (A) may comprise, for example, a coating composition comprising (A1) compounds containing one or more free-radically polymerizable double bonds and further containing at least one other functional group which is reactive in the sense of an addition reaction and/or condensation reaction (examples have been given above), (A2) compounds containing one or more free-radically polymerizable double bonds and further containing at least one other functional group which is reactive in a sense of an addition reaction and/or condensation reaction, the additional reactive functional group being complementary to or reactive toward the additional reactive functional groups of component (A1), (A3) if desired, at least one monomeric, oligomeric and/or polymeric compound containing at least one functional group which is reactive in the sense of an addition reaction and/or condensation reaction toward the functional groups from component (A1) or component (A2) that are present in addition to the free-radically polymerizable double bonds.

Component (A2) carries in each case the groups which are reactive toward or complementary to component (A1). In this context it is possible in each case for different kinds of functional groups to be present in one component. In component (A3) there is a further component available containing functional groups which are reactive in the sense of addition reactions and/or condensation reactions and which are able to react with the functional groups of (A1) or (A2) that are present in addition to the free-radically polymerizable double bonds. Component (A3) contains no free-radically polymerizable double bonds. Examples of such combinations of (A1), (A2), (A3) can be found in WO-A-99/55785

Preferably, component (B) is added to the organic material in an amount from 0.01 to 20%, in particular 0.01 to 10%, for example 0.01 to 5%, relative to the weight of the organic material.

Coating systems include marine coatings, wood coatings, other coatings for metals and coatings over plastics and ceramics. Exemplary of marine coatings are gel coats comprising an unsaturated polyester, a styrene and a catalyst.

The coating is, for example a house paint, or other decorative or protective paint. It may be a paint or other coating that is applied to cement, concrete or other masonry article. The coating may be a water proofer as for a basement or foundation.

As the antimicrobial composition is intended for use in maritime applications as well as near pool areas etc., the composition may be part of a non-skid coating including coatings for stairs, paths and handrails.

The coating composition is applied to a surface by any conventional means including spin coating, dip coating, spray coating, draw down, or by brush, roller or other applicator. A drying or curing period will typically be needed. For impregnating, it is also possible to use pressure impregnation or impregnation without pressure application.

The antimicrobial composition may be part of a polish, such a furniture polish, or a dispersant or surfactant formulation such as a glycol or mineral oil dispersion or other formulation as used in for example wood protection, paper or cardboard protection or the like.

Examples of useful surfactants include, but are not limited to, polyoxyethylene-based surface-active substances, including polyoxyethylene sorbitan tetraoleate (PST), polyoxyethylene sorbitol hexaoleate (PSH), polyoxyethylene 6 tridecyl ether, polyoxyethylene 12 tridecyl ether, polyoxyethylene 18 tridecyl ether, Tween® surfactants, Triton® surfactants, and the polyoxyethlene-polyoxypropylene copolymers such as the Pluronic® and Poloxamer® product series (from BASF). Other matrix-forming components include dextrans, linear PEG molecules (MW 500 to 5,000,000), star-shaped PEG molecules, comb-shaped and dendrimeric, hyperbrached PEG molecules, as well as the analogous linear, star, and dendrimer polyamine polymers, and various carbonated, perfluorinated (e.g., DuPont Zonyl® fluorosurfactants) and siliconated (e.g, dimethylsiloxane-ethylene oxide block copolymers) surfactants (other than those of the present invention).

Given the wide array of applications for the present antimicrobial compositions, the composition may contain one or more other additives such as antioxidants, UV absorbers, benzofuran-2-ones, polyamide stabilizers, metal stearates, nucleating agents, fillers, reinforcing agents, lubricants, emulsifiers, dyes, pigments, dispersants, optical brighteners, flow control agents, flame retardants, antistatic agents, blowing agents, thixotropic agents, adhesion promoters, light stabilizers, curing catalysts, accelerators, inhibitors and the like, such as the materials listed below, or mixtures thereof:
1. Antioxidants
1.1. Alkylated monophenols, for example 2,6-di-tert-butyl-4-methylphenol
1.2. Alkylthiomethylphenols, for example 2,4-dioctylthiomethyl-6-tert-butylphenol
1.3. Hydroquinones and alkylated hydroquinones, for example 2,6-di-tert-butyl-4-methoxyphenol, 2,5-di-tert-butylhydroquinone,
1.4. Tocopherols, for example 1α-tocopherol
1.5. Hydroxylated thiodiphenyl ethers, for example 2, 2′-thiobis(6-tert-butyl-4-methylphenol),
1.6. Alkylidenebisphenols, for example 2, 2′-methylenebis(6-tert-butyl-4-methylphenol),
1.7. O-, N- and S-benzyl compounds, for example 3, 5,3′,5′-tetra-tert-butyl-4,4′-dihydroxydibenzyl ether
1.8. Hydroxybenzylated malonates, for example dioctadecyl-2,2-bis(3,5-di-tert-butyl-2-hydroxybenzyl)malonate,
1.9. Aromatic hydroxybenzyl compounds, for example 1, 3,5-tris(3,5-di-tert-butyl-4-hydroxybenzyl)-2,4,6-trimethylbenzene,
1.10. Triazine compounds, for example 2,4-bis(octylmercapto)-6-(3,5-di-tert-butyl-4-hydroxyanilino)-1,3,5-triazine,
1.11. Benzylphosphonates, for example dimethyl-2,5-di-tert-butyl-4-hydroxybenzylphosphonate
1.12. Acylaminophenols, for example 4-hydroxylauranilide
1.13. Esters of β-(3,5-di-tert-butyl-4-hydroxyphenyl)propionic acid with mono- or polyhydric alcohols
1.14. Esters of β-(5-tert-butyl-4-hydroxy-3-methylphenyl)propionic acid with mono- or polyhydric alcohols
1.15. Esters of β-(3,5-dicyclohexyl-4-hydroxyphenyl)propionic acid with mono- or polyhydric alcohols,
1.16. Esters of 3,5-di-tert-butyl-4-hydroxyphenyl acetic acid with mono- or polyhydric alcohols
1.17. Amides of β-(3,5-di-tert-butyl-4-hydroxyphenyl)propionic acid e.g. N,N′-bis(3,5-di-tert-butyl-4-hydroxyphenylpropionyl)hexamethylenediamide
1.18. Ascorbic acid (vitamin C)
1.19. Aminic antioxidants, for example N,N′-di-isopropyl-p-phenylenediamine
2. UV absorbers and light stabilizers
2.1. 2-(2′-Hydroxyphenyl)benzotriazoles, for example 2-(2′-hydroxy-5′-methylphenyl)-benzotriazole
2.2. 2-Hydroxybenzophenones, for example the 4-hydroxy derivatives.
2.3. Esters of substituted and unsubstituted benzoic acids, for example 4-tert-butyl-phenyl salicylate
2.4. Acrylates, for example ethyl α-cyano-β,β-diphenylacrylate
2.5. Nickel compounds, for example nickel complexes of 2,2′-thio-bis[4-(1,1,3,3-tetramethylbutyl)phenol]
2.6. Sterically hindered amines, for example bis(2,2,6,6-tetramethyl-4-piperidyl)sebacate,
2.7. Oxamides, for example 4, 4′-dioctyloxyoxanilide
2.8. 2-(2-Hydroxyphenyl-1)-1,3,5-triazines, for example 2,4,6-tris(2-hydroxy-4-octyloxyphenyl)-1,3,5-triazine
3. Metal deactivators, for example N,N′-diphenyloxamide,
4. Phosphites and phosphonites, for example triphenyl phosphite
5. Hydroxylamines, for example N,N-dibenzylhydroxylamine
6. Nitrones, for example, N-benzyl-alpha-phenylnitrone
7. Thiosynergists, for example dilauryl thiodipropionate,
8. Peroxide scavengers, for example esters of 6-thiodipropionic acid
9. Polyamide stabilizers, for example copper salts in combination with iodides and/or phosphorus compounds and salts of divalent manganese.
10. Basic co-stabilizers, for example melamine
11. Nucleating agents, for example inorganic substances, such as talcum, metal oxides,
12. Fillers and reinforcing agents, for example calcium carbonate, silicates
13. Other additives, for example plasticisers, lubricants, emulsifiers, pigments, rheology additives, catalysts, flow-control agents, optical brighteners, flameproofing agents, antistatic agents and blowing agents.
14. Benzofuranones and indolinones, for example those disclosed in U.S. Pat. No. 4,325,863; U.S. Pat. No. 4,338,244; U.S. Pat. No. 5,175,312; U.S. Pat. No. 5,216,052; U.S. Pat. No. 5,252,643; DE-A-4316611; DE-A-4316622; DE-A-4316876; EP-A-0589839, EP-A-0591102; EP-A-1291384

The additional additives are added, for example, in concentrations of 0.01 to 10%, relative to the total weight of the material or article.

Incorporation of component (B) and, if desired, further additives into the “bulk” polymeric, organic material is carried out by known methods, for example before or during moulding or else by applying the dissolved or dispersed compounds to the polymeric, organic material, if appropriate with subsequent slow evaporation of the solvent. Component (B) can also be added to the materials in the form of a masterbatch or a colloidal sol or organosol containing for example 5 to 50% by weight of component (B).

Component (B) can also be added before or during polymerisation or before crosslinking.

Component (B) can be incorporated into the material in pure form or encapsulated in waxes, oils or polymers.

Component (B) can also be sprayed or applied as powder onto the material.

The materials thus treated as mentioned above can be used in various forms, for example as films, fibers, ribbons, molded materials, profiles, coatings or as binders for paints, adhesives or cement.

The antimicrobial (e.g. antifouling) composition of the invention may be a coating or a film, a composition for admixing to a material and/or a composition for impregnating a material and/or a product. When the antimicrobial composition is a thermoplastic film which is applied to a surface, for example, by the use of an adhesive or by melt applications including calendaring and co-extrusion, the binder is the thermoplastic polymer matrix used to prepare the film.

When the antimicrobial composition is, e.g., a coating, it may be applied as a liquid solution or suspension, a paste, gel, oil or the coating composition may be a solid, for example a powder coating which is subsequently cured by heat, UV light or other method. As the antimicrobial composition of the invention may be a coating or a film, the binder can be comprised of any polymer used in coating formulations or film preparation.

Examples of materials to which an acid compound or mixture of acid compounds or composition comprising such acid or mixture can be applied as a coating or in bulk are especially organic materials, preferably synthetic materials, preferably made from polymers (which may also be used in forming articles comprising them or especially consisting of them), such as thermoplastic, elastomeric, inherently crosslinked or crosslinked polymers, or mixtures of precursors thereof (such as monomers), especially synthetic materials from those listed below, where the term “materials comprising (especially consisting of) synthetic polymers or polymer precursors” especially relates to the embodiments marked with an asterisk:
1.* Polymers of mono- and di-olefins, for example polypropylene, polyisobutylene, polybutene-1, poly-4-methylpentene-1, polyisoprene or polybutadiene and also polymerisates of cyclo-olefins, for example of cyclopentene or norbornene; and also polyethylene (which may optionally be crosslinked), for example high density polyethylene (HDPE), high density polyethylene of high molecular weight (HDPE-HMW), high density polyethylene of ultra-high molecular weight (HDPE-UHMW), medium density polyethylene (MDPE), low density polyethylene (LDPE), and linear low density polyethylene (LLDPE), (VLDPE) and (ULDPE).

Such polyolefins, i.e. the polymers of monoolefins exemplified above in the present paragraph, preferably polyethylene and polypropylene, can be prepared by different, e.g. preferably the following methods:
(i) radical polymerization (normally under high pressure and at elevated temperature); or
(ii) catalytic polymerization using a catalyst that normally contains one or more than one metal of groups Ivbm Vb, Vib or VII of the Periodic Table. These metals often have more than one ligand.
2.* Mixtures of the polymers mentioned under 1).
3.* Copolymers of mono- and di-olefins with one another or with other vinyl monomers, for example ethylene/propylene copolymers.
4.* Hydrocarbon resins (for example C₅-C₉) including hydrogenated modifications thereof (e.g. tackifiers) and mixtures of polyalkylenes and starch.
   (*Homopolymers and copolymers from 1. to 4. may have any stereostructure including syndiotactic, isotactic, hemi-isotactic or atactic; where atactic polymers are preferred.
   Stereoblock polymers are also possible.)
5.* Polystyrene, poly(p-methylstyrene), poly(α-methylstyrene).
6.* Aromatic hompoolymers and copolymers derived from vinyl aromatic monomers including styrene. Stereoblock polymers are also included.
6a*. Copolymers including aforementioned vinyl aromatic monomers and comonomers
6b.* Hydrogenated aromatic polymers derived from hydrogenation of polymers mentioned under 6.)
6c.* Hydrogenated aromatic polymers derived from hydrogenation of polymers mentioned under 6a.).
   (*Homopolymers and copolymers may have any stereostructure including syndiotactic, isotactic, hemi-isotactic or atactic; where atactic polymers are preferred. Stereoblock polymers are also included.)
7.* Graft copolymers of vinyl aromatic monomers such as styrene or α-methylstyrene, for example styrene on polybutadiene as well as mixtures thereof with the copolymers listed under 6)
8.* Halogen-containing polymers such as polychloroprene, especially polymers of halogen-containing vinyl compounds, for example polyvinyl chloride as well as copolymers thereof such as vinyl chloride/vinylidene chloride, vinyl chloride/vinyl acetate or vinylidene chloride/vinyl acetate copolymers.
9.* Polymers derived from α,β-unsaturated acids and derivatives thereof such as polyacrylates and polymethacrylates.
10.* Copolymers of the monomers mentioned under 9) with each other or with other unsaturated monomers, for example acrylonitrile/butadiene copolymers.
11.* Polymers derived from unsaturated alcohols and amines or the acyl derivatives or acetals thereof, for example polyvinyl alcohol; as well as their copolymers with olefins mentioned in 1) above.
12.* Homopolymers and copolymers of cyclic ethers such as polyalkylene glycols, polyethylene oxide, polypropylene oxide or copolymers thereof with bisglycidyl ethers.
13.* Polyacetals such as polyoxymethylene and those polyoxymethylenes which contain ethylene oxide as a comonomer; polyacetals modified with thermoplastic polyurethanes, acrylates or MBS.
14.* Polyphenylene oxides and sulfides, and mixtures of polyphenylene oxides with styrene polymers or polyamides.
15.* Polyurethanes derived from hydroxyl-terminated polyethers, polyesters or polybutadienes on the one hand and aliphatic or aromatic polyisocyanates on the other, as well as precursors thereof.
16.* Polyamides and copolyamides derived from diamines and dicarboxylic acids and/or from aminocarboxylic acids or the corresponding lactams, for example polyamide 4, polyamide 6, and also block copolymers of the aforementioned polyamides with polyolefins, olefin copolymers, ionomers or chemically bonded or grafted elastomers; or with polyethers, e.g. with polyethylene glycol, polypropylene glycol or polytetramethylene glycol; as well as polyamides or copolyamides modified with EPDM or ABS; and polyamides condensed during processing (RIM polyamide systems).
17.* Polyureas, polyimides, polyamide-imides, polyetherimids, polyesterimids, polyhydantoins and polybenzimidazoles.
18.* Polyesters derived from dicarboxylic acids and diols and/or from hydroxycarboxylic acids or the corresponding lactones, for example polyethylene terephthalate, as well as block copolyether esters derived from hydroxyl-terminated polyethers; and also polyesters modified with polycarbonates or MBS.
19.* Polycarbonates and polyester carbonates.
20.* Polyketones.
21.* Polysulfones, polyether sulfones and polyether ketones.
22.* Crosslinked polymers derived from aldehydes on the one hand and phenols, ureas and melamines on the other hand, such as phenol/formaldehyde resins, urea/formaldehyde resins and melamine/formaldehyde resins.
23.* Drying and non-drying alkyd resins.
24.* Unsaturated polyester resins derived from copolyesters of saturated and unsaturated dicarboxylic acids with polyhydric alcohols and vinyl compounds as crosslinking agents, and also halogen-containing modifications thereof of low flammability.
25.* Crosslinkable acrylic resins derived from substituted acrylates, for example epoxy acrylates, urethane acrylates or polyester acrylates.
26.* Alkyd resins, polyester resins and acrylate resins crosslinked with melamine resins, urea resins, isocyanates, isocyanurates, polyisocyanates or epoxy resins.
27.* Crosslinked epoxy resins derived from aliphatic, cycloaliphatic, heterocyclic or aromatic glycidyl compounds.
28.* Natural polymers such as cellulose (which is excluded where only coating or impregnation of materials or articles made from synthetic polymers is mentioned, except if in isolated or refined form not present in nature and other than paper, e.g. in the form of textile yarns or fabrics), or preferably rubber, gelatin and especially chemically modified homologous derivatives thereof.
29.* Blends of tow or more of the aforementioned polymers (polyblends).
30.* Pre-polymeric monomers or oligomers of two or more of the aforementioned polymers or blends;
31.* Aqueous emulsions of natural and/or preferably *synthetic rubber, e.g. natural latex or preferably *latices of carboxylated styrene/butadiene copolymers.

### Other Materials are

32. Naturally occurring and synthetic* organic materials which are pure monomeric compounds or mixtures of such compounds.
33.* Sols, especially organosols, as stable liquid suspensions of colloidal nano-particles in a diluent, a reactive (e.g. crosslinking) diluent or in a polymerizable or crosslinking monomer, or in a mixture of all.

“Other monomeric compound” may, for example, refer to non-polymer materials, such as powders, dispersions or solutions of drugs (with or without carrier materials) or other smaller, non polymer chemical entities, e.g. with relative molecular weights up to 2000, preferably 1000 or lower.

The surface being coated (including laminated) and/or impregnated is the surface of any substrate (other word for material or product used herein) exposed to biofouling conditions. The substrate can be an inorganic or organic substrate, for example, based on a metal or metal alloy; a thermoplastic, elastomeric, inherently crosslinked or crosslinked polymer as described above; a natural polymer such as wood or rubber; a ceramic material; glass; a yarn; a non-woven material (e.g. for diapers or the like, such as PP non-wovens); paper; leather or other textile (e.g. for clothing, for technical purposes, for canvas or the like, e.g. from cotton, wool, latex and/or synthetic fibres.

Compositions with one or more acids of formula III are preferably applied to materials other than fabrics or paper.

The substrate may also be, for example, non-metal inorganic surfaces such as silica, silicon dioxide, titanium oxides, aluminum oxides, iron oxides, carbon, silicon, various silicates and sol-gels, masonry, and composite materials such as fiberglass and plastic lumber (a blend of polymers and wood shavings, wood flour or other wood particles).

The inorganic or organic substrate is, for example, a metal or metal alloy, a thermoplastic, elastomeric, inherently crosslinked or crosslinked polymer, a ceramic material or a glass.

The substrate may be a multi-layered article comprised of the same or different components in each layer. The surface coated, laminated and/or impregnated may be the exposed surface of an already applied coating or laminate.

The inorganic or organic substrate to be coated (this term including laminated) and/or impregnated can be in any solid form.

For example, polymer substrates may be plastics in the form of films, injection-molded articles, extruded workpieces, fibres, felts, non-woven or woven fabrics.

For example molded or extruded polymeric articles used in construction or the manufacture of durable goods such as siding, fascia and mailboxes can all benefit from the present method for stabilizer replenishment.

Plastics which would benefit from the uses or methods according to the invention include, but are not limited to, plastics used in construction or the manufacture of durable goods or machine parts, including outdoor furniture, boats, siding, roofing, glazing, protective films, decals, sealants, composites like plastic lumber and fiber reinforced composites, functional films including films used in displays as well as articles constructed from synthetic fibers such as awnings, fabrics such as used in canvas or sails and rubber articles such as outdoor matting and other uses cited in this disclosure. Exemplary of such plastics are polypropylene, polyethylene, PVC, POM, polysulfones, styrenics, polyamides, urethanes, polyesters, polycarbonate, acrylics, butadiene, thermoplastic polyolefins, ionomers, unsaturated polyesters and blends of polymer resins including ABS, SAN and PC/ABS.

The invention also provides a method of preventing biofouling of surfaces and/or materials, wherein an acid compound according to the invention is incorporated into a coating formulation or film which is then applied to the surface of an article.

Examples of applications of the antimicrobial compositions of the instant invention are surface coatings, protective paints, impregnation compositions, other coatings and laminates applied to vulnerable surfaces, for example, the hulls of ships, surfaces of docks or the inside of pipes in circulating or pass-through water systems, walls exposed to rain water, walls of showers, roofs, gutters, pool areas, saunas, floors and walls exposed to damp environs such as basements or garages, the housing of tools and outdoor furniture.

For example, the antimicrobial compositions of the instant invention are found, among other places, on the surfaces and/or in the materials of: boat hulls, docks, buoys, drilling platforms, ballast water tanks, machines, machine parts, recreational, air conditioning systems, ion exchangers, process water systems, other industrial water systems, solar-powered units, heat exchangers, sump pumps, drainage systems, roofing, basements, walls, facades, greenhouses, sheds, storage areas, awnings, garden fencing, wood protection, tent roof material, fabrics, outdoor furniture, door mats, public conveniences, bathrooms, showers, swimming pools, saunas, jointing, sealing compounds, public conveyances, locker rooms, and the like.

Process water includes any process water stream which is used for heating or cooling purposes in closed or open circulating systems.

In order to be active against microorganism and colonization by organisms, an acid compound or an antimicrobial composition according to the invention can, alternatively or in addition to being used for a coating and/or for impregnating, also be admixed to materials or intermediate products used to form products or articles, e.g. to oligomer- and or pre-polymer mixtures or melts (e.g. for extrusion or molding) or components used to form articles from natural or especially synthetic materials, or e.g. to glue or other binding materials used to bind wood or other chips in the production of pressboard or imitation pressboard, to adhesives, cements or other mortar or concrete components, to mortars, to resins, to solutions or the like.

Where hereinbefore reference is made to acids, acid compounds, acid compound mixtures, compositions or the like, also the corresponding use, method or process of use is included, especially of such compositions or agents comprising an acid compound of the formula I or II.

The antimicrobial agents according to the present invention are also suitable as antimicrobials in cosmetic formulations, home care compositions or pharmaceutical compositions, collectively referred to as antimicrobial preparations hereinafter, especially to make use of their antimicrobial or preservative effect, further also for their anti-adhesion effects. For example, the antimicrobial agents cosmetic personal care applications such as deodorants, skin, hair and oral care products and rinse off products in home care applications for cleaning and disinfection of hard surfaces and fabric care applications such as liquid detergents and softeners, or in cosmetic formulations or pharmaceutical compositions. In each case, the antimicrobial preparations may, in addition to the antimicrobial agent according to the present invention, comprise one or more than one further antimicrobial agent.

The antimicrobial preparations can be prepared by physically mixing the antimicrobial agent(s) with an adjuvant using customary methods, for example by simply stirring together the individual components, especially by making use of the dissolution properties of already known antimicrobial agents.

The antimicrobial preparations, especially cosmetic formulations or pharmaceutical compositions, usually contain from 0.01-40% by weight of an acid compound or an acid compound mixture according to the invention. Highly preferred is an amount of 0.01 to 5% by weight.

Further antimicrobials which can additionally be used in the present invention are known to those skilled in the art.

Combinations with chelating agents can also improve the antimicrobial activity of the antimicrobial agents of the present invention.

The antimicrobial preparations of the present invention can in addition comprise from about 0.05% to about 10% by weight of an anionic surfactant.

Non-limiting examples of anionic lathering surfactants useful in the compositions of the present invention are disclosed in McCutcheon's, Detergents and Emulsifiers, North American edition (1990), published by The Manufacturing Confectioner Publishing Co.; McCutcheon's, Functional Materials, North American Edition (1992); and U.S. Pat. No. 3,929,678, to Laughlin et al., issued Dec. 30, 1975, all of which are incorporated by reference.

The antimicrobial preparations of the present invention may further comprise a non-ionic surfactant. Typical nonionic surfactants are condensated products of ethylene oxide with various reactive hydrogen-containing compounds reactive therewith having long hydrophobic chains.

The antimicrobial preparations of the present invention may also comprise a proton donating agent, most preferably from about 1% to about 5% by weight.

In order to achieve the mildness required of the antimicrobial preparation of the present invention, optional ingredients to enhance the mildness to the skin can be added. These ingredients include cationic and nonionic polymers, co-surfactants, moisturizers and mixtures thereof.

Another group of mildness enhancers are lipid skin moisturizing agents which provide a moisturizing benefit to the user when the lipophilic skin moisturizing agent is deposited to the user's skin. When in antimicrobial personal cleansing compositions herein lipophilic skin moisturizing agents are used, they are employed at a level of most preferably from about 0.5% to about 5% by weight of the composition.

A wide variety of lipid type materials and mixtures of materials are suitable for use in the antimicrobial preparations of the present invention. Preferably, the lipophilic skin conditioning agent is selected from the group consisting of hydrocarbon oils and waxes, silicones, fatty acid derivatives, cholesterol, cholesterol derivatives, di- and tri-glycerides, vegetable oils, vegetable oil derivatives, liquid nondigestible oils such as those described in U.S. Pat. No. 3,600,186 to Mattson; Issued Aug. 17, 1971 and U.S. Pat. Nos. 4,005,195 and 4,005,196 to Jandacek et al; both issued Jan. 25, 1977, all of which are herein incorporated by reference, or blends of liquid digestible or nondigestible oils with solid polyol polyesters such as those described in U.S. Pat. No. 4,797,300 to Jandacek; issued Jan. 10, 1989; U.S. Pat. Nos. 5,306,514, 5,306,516 and 5,306,515 to Letton; all issued Apr. 26, 1994, all of which are herein incorporated by reference, and acetoglyceride esters, alkyl esters, alkenyl esters, lanolin and its derivatives, milk tri-glycerides, wax esters, beeswax derivatives, sterols, phos-pholipids and mixtures thereof. Fatty acids, fatty acid soaps and water soluble polyols are specifically excluded from our definition of a lipophilic skin moisturizing agent.

When a lipophilic skin moisturizing agent is employed as the mildness enhancer in the antimicrobial preparations herein, a stabilizer may also be included at a level preferably from about 0.1% to about 5% by weight of the antimicrobial preparation. The stabilizers used herein are not surfactants. The stabilizers provide improved shelf and stress stability.

The antimicrobial preparations of the present invention can comprise a wide range of optional ingredients. The CTFA International Cosmetic Ingredient Dictionary, Sixth Edition, 1995, which is incorporated by reference herein in its entirety, describes a wide variety of nonlimiting cosmetic and pharmaceutical ingredients commonly used in the skin care industry suitable for use in the antimicrobial preparations of the present invention. Nonlimiting examples of functional classes of ingredients are described at page 537 of this reference.

Examples of these functional classes include: abrasives, anti-acne agents, anticaking agents, antioxidants, binders, biological additives, bulking agents, chelating agents, chemical additives, colorants, cosmetic astringents, cosmetic biocides, denaturants, drug astringents, emulsifiers, external analgesics, film formers, fragrance components, humectants, opacifying agents, plasticizers, preservatives, propellants, reducing agents, skin bleaching agents, skin-conditioning agents (emollient, humectants, miscellaneous, and occlusive), skin protectants, solvents, foam boosters, hydrotropes, solubilizing agents, suspending agents (nonsurfactant), sunscreen agents, ultraviolet light absorbers, and viscosity increasing agents (aqueous and nonaqueous). Examples of other functional classes of materials useful herein that are well known to one of ordinary skill in the art include solubilizing agents, sequestrants, and keratolytics, and the like.

The antimicrobial agents of the present invention can be used as ingredients in a wide variety of cosmetic preparations. There come into consideration, for example, especially one or more of the following preparations: skin-care preparations, bath preparations, cosmetic personal care preparations, foot-care preparations; light-protective preparations, skin-tanning preparations, depigmenting preparations, insect-repellents, deodorants, antiperspirants, preparations for cleansing and caring for blemished skin, hair-removal preparations in chemical form (depilation), shaving preparations, fragrance preparations or cosmetic hair-treatment preparations, or the like.

The final formulations may exist in a wide variety of presentation forms, for example in the form of liquid preparations as a W/O, O/W, O/W/O, W/O/W or PIT emulsion and all kinds of microemulsions, in the form of a gel, an oil, a cream, milk or lotion, a powder, a lacquer, a tablet or make-up, a stick, a spray or an aerosol, a foam, or a paste.

The antimicrobial agents of the present invention (especially those described as novel or regarding (methods of) their use with provisos as defined herein) can also be used against oral bacteria and to improve anti-plaque effectiveness, anti-gingivitis activities and to help to reduce paradontitis.

The activity can be improved by combinations with other antimicrobial actives or anti-plaque and anti-gingivits actives such as for example chlorhexidine or phenolic substances such as 2,4,4′ trichloro 2′-hydroxy diphenylether.

Typical oral preparations containing an antimicrobial agent of the present invention alone or in combinations with one or more of the above mentioned antimicrobials and anti-plaque agents are e.g. mouthrinses, semi-solids such as toothpastes or gel dentifrices, chewing gums or solid lozenge or the like.

Furthermore, an oral composition may comprise:

polishing agents, humectants, water, natural or synthetic thickener or gelling agent, alcohol such as ethanol or isopropanol, organic surface-active agents which can be cationic, anionic or non-ionic, flavoring agents, sweetening agents, agents used to diminish teeth sensitivity, whitening agents such as urea peroxide and hydrogen peroxide, preservatives such as sodium benzoate, substances which release fluoride ions to protect against caries, and/or other agents such as chlorophyll compounds and/or ammoniated materials such as urea, diammonium phosphate and mixtures thereof.

Antibacterial enhancing agents may be included in the oral composition.

Preferably, the antibacterial enhancing agent is an anionic polymer comprising a chain or backbone containing repeating units each preferably containing at least one carbon atom and preferably at least one directly or indirectly pendent, monovalent delivery-enhancing group and at least one directly or indirectly pendent monovalent retention-enhancing group geminally, vicinally or less preferable otherwise bonded to atoms, preferably carbon, in the chain.

The antimicrobial agents of the present invention can also be used as additives in laundry detergent and/or fabric care compositions, especially under consideration of the provisos regarding their use. The laundry detergent and/or fabric care compositions of the present invention preferably further comprise a detergent ingredient selected from cationic, anionic and/or nonionic surfactants and/or bleaching agent.

The antimicrobial laundry detergent and/or fabric care compositions according to the invention can be liquid, paste, gels, bars, tablets, spray, foam, powder or granular forms. Granular compositions can also be in “compact” form, the liquid compositions can also be in a “concentrated” form.

The compositions of the invention may, for example, be formulated as hand and machine laundry detergent compositions.

When formulated as compositions suitable for use in a laundry machine washing method, the laundry detergent and/or fabric care compositions of the invention preferably comprise both a surfactant and a builder compound and additionally one or more detergent components preferably selected from organic polymeric compounds, bleaching agents, additional enzymes, suds suppressors, dispersants, lime-soap dispersants, soil suspension and anti-redeposition agents and corrosion inhibitors. Laundry compositions can also contain softening agents, as additional detergent components.

The laundry detergent and/or fabric care compositions of the present invention may also contain cationic fabric softening components which include the water-insoluble quaternary-ammonium fabric softening actives or the corresponding amine precursor, the most commonly used having been di-long alkyl chain ammonium chloride or methyl sulfate.

The laundry detergent and/or fabric care compositions of the present invention may also contain ampholytic, zwitterionic, and semi-polar surfactants.

The laundry detergent and/or fabric care compositions may further comprise one or more enzymes which provide cleaning performance, fabric care and/or sanitisation benefits. The antimicrobial laundry detergent compositions according to the present invention may further comprise a builder system. Any conventional builder system is suitable for use herein. The antimicrobial laundry detergent and/or fabric care compositions herein may also optionally contain one or more iron and/or manganese chelating agents.

The antimicrobial laundry detergent compositions compositions herein may also contain water-soluble methyl glycine diacetic acid (MGDA) salts (or acid form) as a chelant or cobuilder useful with, for example, insoluble builders such as zeolites, layered silicates and the like.

Another optional ingredient is a suds suppressor, exemplified by silicones, and silicasilicone mixtures.

Other components such as soil-suspending agents, soil-release agents, optical brighteners, abrasives, bactericides, tarnish inhibitors, colouring agents, and/or encapsulated or non-encapsulated perfumes may be employed.

The laundry detergent and/or fabric care composition of the present invention can also contain dispersants: Suitable water-soluble organic salts are the homo- or co-polymeric acids or their salts, in which the polycarboxylic acid comprises at least two carboxyl radicals separated from each other by not more than two carbon atoms.

The laundry detergent and/or fabric care compositions of the present invention can also include compounds for inhibiting dye transfer from one fabric to another of solubilized and suspended dyes encountered during fabric laundering operations involving coloured fabrics.

The antimicrobial agents according to the present invention can also be applied to textiles, which treatment can be carried out before dyeing of the textiles, during dyeing or after dyeing (as an after-treatment). The application of the antimicrobials can, for example, be carried out by an exhaustion process, padding, spraying or by foam application.

The antimicrobial agents according to the present invention may be applied as aqueous formulation in diluted, solubilized, emulsified or dispersed form.

Such aqueous formulations can additionally comprise a small amount of an organic solvent, a surfactant, a dispersant, and/or an emulsifier.

Padding can be carried out according to conventional padding processes. For example, the textile material is passed through an aqueous liquor comprising the antimicrobial agent, the textile material is squeezed to a defined liquor pick-up rate and then a fixation step is carried out, preferably a heat treatment.

The amount of the antimicrobial agent according to the present invention in the aqueous liquor (padding liquor) is usually 0.001% to 10% by weight, an amount of the antimicrobial agent of 0.01% to 5% by weight is preferred.

The fixation step is usually carried out by a heat treatment, for example at a temperature of 60 to 150° C., especially 90 to 150° C.

The padding process is usually carried out as a continuous process wherein the textile material is continuously passed through the aqueous liquor containing the antimicrobial agent.

The application of the antimicrobial agent according to the exhaustion process is usually carried out from an aqueous liquor, at a pH value of from 2 to 9, from 4 to 7, and a temperature from 50 to 100° C. and especially from 80 to 100° C. The liquor ratio selected can vary within a wide range, for example from 1:5 to 1:50, preferably from 1:5 to 1:30.

The amounts in which the antimicrobial agents are used in the dye baths may vary within wide limits; amounts of from 0.01 to 10% by weight, especially from 0.01 to 5% by weight, based on the goods to be treated, have generally proved advantageous.

Spraying can be carried out according to conventional spraying processes. According to these processes aqueous liquids comprising the antimicrobial agent according to the present invention are sprayed onto the textile material. The amount of the antimicrobial agent in the aqueous liquor is usually 0.001% to 10% by weight, especially 0.01% to 10% by weight, based on the weight of the aqueous liquor. An amount of the antimicrobial agent of 0.1% to 10% by weight is preferred. Such spraying processes are especially suitable for applying the antimicrobial agent to textile materials like carpets. According to such preferred processes a plurality of spray nozzles are disposed in a spray line transverse to the direction of movement of, for instance, the carpet. The antimicrobial agent is applied as an aqueous liquor by the spray nozzles, for example by virtue of pressure.

After spraying, usually a fixation step is carried out, which can be performed by a heat treatment as given above for the padding process.

Spraying can also be used to apply the antimicrobial agent in form of an aqueous liquor to surfaces of textile materials including leather, like sofas or shoes.

The antimicrobial agent according to the present invention can also be applied to the textile material by foam application. As to this application all of the above conditions and preferences given above for the spraying process apply. However, the antimicrobial agent according to the present invention is applied in form of an aqueous foam which usually in addition contains a foam stabiliser and may comprise other customary additives. Such a process is also especially suitable for treating carpets.

Exhaustion, padding, spraying or foam applications can be carried out by applying the anti-microbial agent to the textile material together with dyestuffs (for example in a dyeing process) or in other textile related processes, like finishing processes. It is preferred to carry out the treatment with the antimicrobial agents in the presence of dyestuffs.

If these processes are carried out without the presence of dyestuffs it is preferred to apply the antimicrobial agent in a finishing process.

The use of some polymeric as well as oligomeric substances that are commonly used in the textile industries, can help to further improve the durability of the desirable antimicrobial efficacy. Such substances include, but are not limited to, resin finishings that provide easy care and/or other properties to various textile materials, softeners, coating materials, fixation agents and/or other finishing agents such as hydrophilic and hydrophobic agents, flame retardant etc. It is obvious, from economic as well as process convenience perspective that the antimicrobial treatment could be carried out together with many other different types of treatments that are found in the textile industries.

The application of the antimicrobial agent according to the present invention can also be carried out in a dyeing process. For such processes the above conditions and preferences apply. Suitable dyes are disperse dyes, basic dyes, acid dyes, direct dyes or reactive dyes. Reactive dyes are especially suitable for natural polyamide- or cellulose-containing textile materials. Direct dyes are especially suitable for cellulose-containing textile materials. The dyes may belong to different dye classes, including acridone, azo, anthraquinone, coumarin, formazane, methine, perinone, naphthoquinone-imine, quinophthalone, styryl or nitro dyes. Mixtures of dyes may also be used.

When using the antimicrobial agent in a dyeing process, the procedure can be such that the textile material is first treated with these compounds and then dyeing is carried out or, preferably, the textile material is treated simultaneously with the antimicrobial agent and the dye. The application of the antimicrobial agent can, however, also be effected subsequently to the previously prepared dyeing.

After the dyeing process including the application of the antimicrobial agent according to the present invention the textile material can be subjected to a fixation step, like a heat treatment as given above.

Textile materials which can be treated with the antimicrobial agents are materials comprising, for example, natural or synthetic polyamide (like wool, silk, nylon), polyurethane, polyester, polypropylene, polyethylene, polyacrylonitrile and cellulose-containing textile materials of all kinds, for example natural cellulose fibres, such as cotton, linen, jute and hemp, and also viscose staple fibre and regenerated cellulose; or blends of the above fibre materials, like polyacrylonitrile/polyester, polyamide/polyester, polyester/cotton, polyester/viscose and polyester/wool.

Preferred textile materials are those comprising wool, synthetic polyamide, polyester, polypropylene, polyethylene, and cellulose-containing textile materials, preferably cotton or wool and especially cotton.

The textile material can be in different forms of presentation, as woven or knitted fabrics or as piece goods such as knitgoods, nonwoven textiles, carpets, yarn or staple fibres. Preferred are nonwoven textile materials and especially carpets.

Numerous end use articles can be named for the treated fabrics or products made from the treated materials. Examples include but are not limited to carpets and rags, pillow cases, bed linings, bed sheets, matrices and matrices ticking, curtains, duvet and duvet cases, upholsteries, socks and garments.

The antimicrobial and (especially with regard to colonization by organisms, especially microorganisms) anti-adhesive properties of the acid compounds of the present invention can be determined according to standard procedures, e.g. by the methods mentioned in the Examples. Such assays show a good to very good antimicrobial activity of the acid compounds according to the invention.

### PREFERRED EMBODIMENTS OF THE INVENTION

The invention relates preferably to those embodiments where one or more of the more general definitions or symbols is/are replaced by a more specific definition given above or below.

Preferably, the invention relates to the uses, methods or processes mentioned herein where only an acid compound or acid compounds of the formula III are present to achieve an antimicrobial effect (that is, as antimicrobials).

Also preferably, in the case of acid compounds only with (=represented by) acids of the formula III the use is limited to the addition to pharmaceuticals.

In the case of acid compounds with acids of the formula II only, the processes, methods or uses are preferably limited to achieve an antimicrobial and/or a preservative effect, more preferably to achieve an antimicrobial effect.

Acids of the formula III are preferably not applied to leather, skin, hair, teeth, fabrics and paper, coatings, inorganic materials, polyester resins, epoxy resins, urethane resins and cosmetics. Acids of the formula II are preferably not applied to coatings, inorganic materials, polyester resins, epoxy resins, urethane resins and cosmetics.

Acids carrying as part of Q functional groups P(O)(OH)₂, as well as the corresponding acids and compositions comprising them, are especially preferred.

The present invention also preferably relates to compositions comprising an acid of the formula I, II and/or III according to the invention falling under the preferred embodiments of such acid, preferably with one or more other additives, preferably the compositions as mentioned above.

The invention relates also to compositions according to the invention comprising a preferred acid compound of the invention especially as described in the preferred embodiments or in the (especially dependent) claims as antimicrobial agent and/or the use (or a method of using) according to the invention of a preferred acid compound of the invention especially as described herein or in the (especially dependent) claims or a composition comprising especially a preferred acid compound of the invention as described herein or in the (especially dependent) claims as antimicrobial agent as antimicrobial agent, comprising administering a composition or an acid compound according to the invention to a material or surface of a product or object. Where required, further steps, such as molding, curing or the like can follow. Administering may also take place by impregnation of an otherwise completed product, an intermediate product and/or a material.

The invention especially refers to the processes, methods, uses, acid compounds and compositions given in the claims, especially in the dependent claims, which are incorporated by reference herein, and/or to the novel processes, methods, uses, acid compounds and compositions given in the Examples.

The following examples illustrate the invention without limiting the scope:

Where percentages are given, they refer to percent by weight. Table of Example compounds

Example

| | |
|---|---|
| 1 | 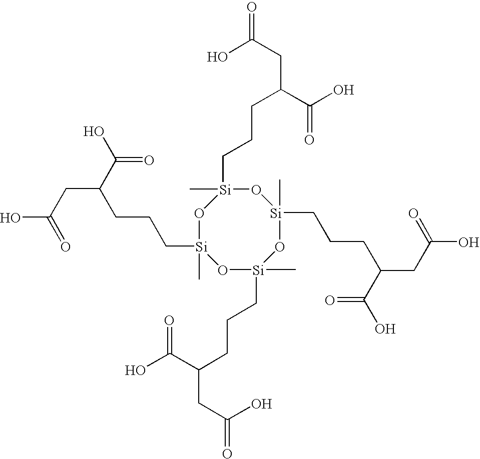 |
| 2 | 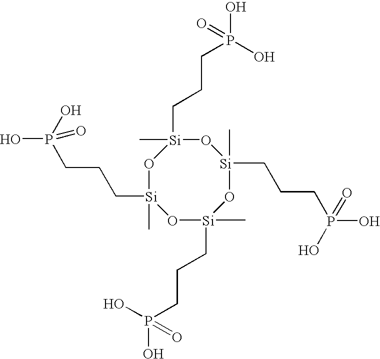 |
| 3 | 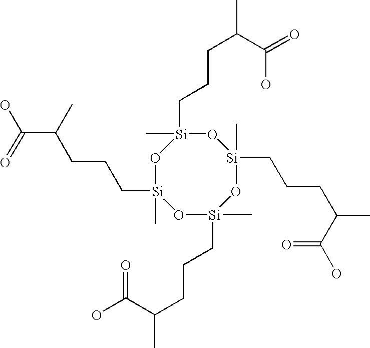 |
| 4 | 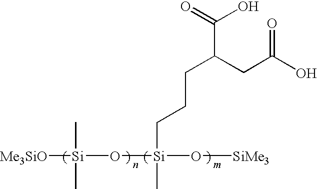 |
| 5 | 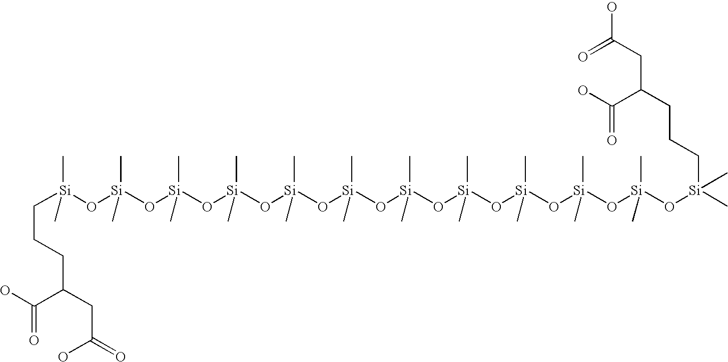 |

The compounds of the examples are prepared as follows:

### Example 1

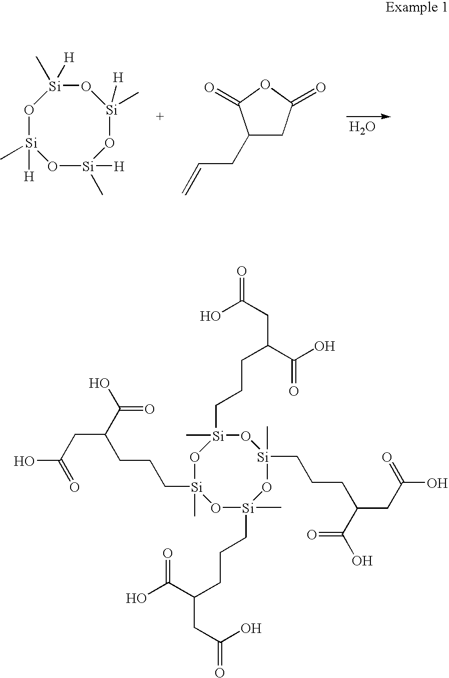

A solution of 3.12 g (13 mmol) of tetramethylcyclotetrasiloxane in 10 ml of toluene is added dropwise slowly at 80° C. to a solution of 8.74 g (62 mmol) allylsuccinic anhydride and a drop of Karstedt-catalyst (di-platinum-tris(1,3-divinyl-1,1,3,3-tetramethyldisiloxane) catalyst) in 10 ml of toluene. The reaction mixture is then kept at 90° C. for 12 h. After evaporation of the solvent, the excess of the anhydride is removed by heating the mixture at 80° C./10-2 mbar for at least 12 h.

For subsequent hydrolysation of the reaction product, the resulting viscous oil is dissolved in 10 ml of toluene and refluxed for 12 h with 5 ml of water. After azeotropic distillation, the reaction product (compound Example 1) is obtained as a highly viscous oil.

Product properties of Example 1: ¹H-NMR (DMSO-d₆[ppm]): 0.20 (s, 3H, SiCH₃), 0.50 (m, 2H, SiCH₂), 1.20-1.65 (m, 4H, CH₂CH₂), 2.30-2.70 (m, 3H, COCHCH₂CO), 11.9 (s, 2H, COOH); LC/ESMS m/z [M-1]=872

### Example 2

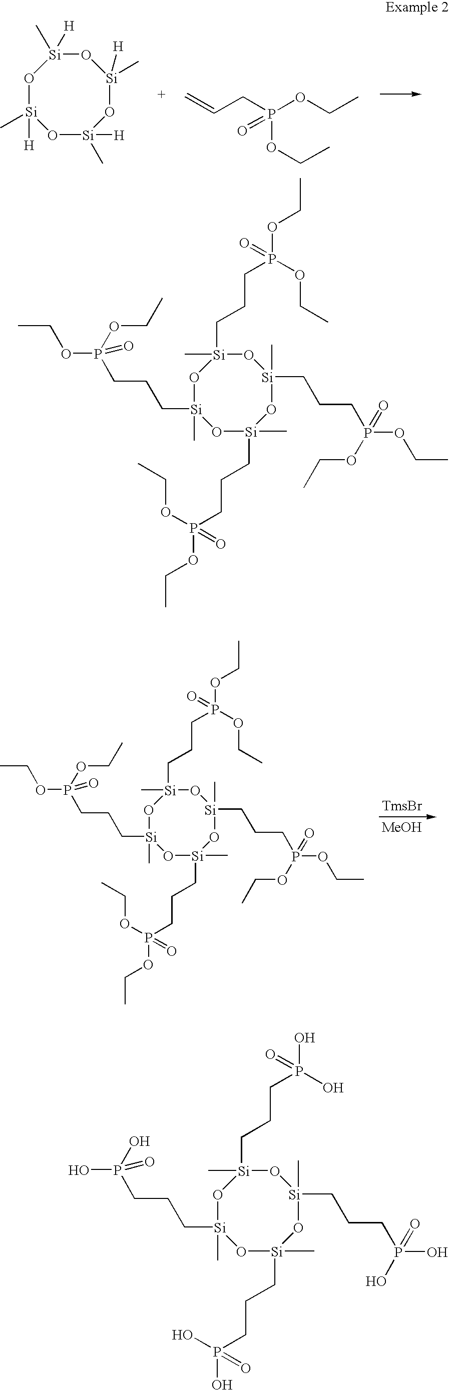

A solution of 2.41 g (10 mmol) of tetramethylcyclotetrasiloxane in 5 ml of toluene is added dropwise slowly at 90° C. to a solution of 7.13 g (40 mmol) allylphosphonic acid diethylester and 3 drops of Karstedt-catalyst in 10 ml of toluene. The reaction mixture is then kept at 90° C. for 16 h. After evaporation of the solvent the residue is used for the subsequent reaction without further work up.

For ester cleavage, 4.77 g (5 mmol) of the ester obtained as before is dissolved in 5 ml of dry acetonitrile, 7.66 g (50 mmol) of bromotrimethylsilane is added and the mixture is stirred for 24 h at room temperature. After removal of the volatiles, the residue is refluxed for 1 h in methanol to obtain the product after removal of the solvent as a light brown powder.

Product properties of Example 2: 1H-NMR (Aceton-d₆, [ppm]): 0-0.2 (m, 3H, CH₃), 0.5-0.7 (m, 2H, SiCH₂), 1.45-1.85 (m, 4H, CH₂), 10.4 (s, 2H, POH)

### Example 3

The compound of Example 3 is prepared in analogy to that of Example 1 by hydrosilylation of tetramethylcyclotetrasiloxane with methylpentenoic acid ethylester and subsequent ester hydrolysis with diluted hydrochloric acid.

Product properties of Example 3: ¹H-NMR (Aceton-d₆[ppm]): —0.05-0.05 (m, 3H, SiCH₃), 0.45 (m, 2H, SiCH₂), 1.00 (d, 3H, CH₃), 1.35/1.60 (m, 4H, CH₂CH₂), 2.30 (m, 1H, CH)

The compounds of Examples 4 and 5 are prepared analogously to the methods described in the preceding Examples or by methods described herein.

### Microbiological Activity of the Compounds

### Example 6

### Microbicidal Use and Activity

Microbicidal activity of different anionic siloxanes with carboxyl groups is tested according to European Standard EN1040. A bacterial suspension with a cell count of about 10⁷ cfu/ml (cfu stands for colony-forming units) is contacted with appropriate concentrations of the specific substances and the residual cell count is determined after incubation times of 5 and 30 min. at room temperature under continuos stirring. Staphylococcus aureus (ATCC 5638, American Type Culture Collection, Manassas, USA) is tested as gram+ (gram positive) and Escherichia coli (ATCC 10535, American Type Culture Collection) as gram-organism. The results are given as a log reduction at each incubation time in comparison to a water control in the following tables (a) and (b):

TABLE (a)

| | | log | | | log | |
|---|---|---|---|---|---|---|
| Test | | reduction | | | log reduction | |
| concentration | S. aureus | | 30 | E. coli | 5 | 30 |
| 1% | cfu/mL | 5 min. | min. | cfu/mL | min. | min. |
| Inoculum | 1.12E+08 | | | 3.23E+08 | | |
| H₂O reference 5′ | 1.56E+07 | | | 1.94E+07 | | |
| H₂O reference 30′ | 1.30E+07 | | | 1.78E+07 | | |
| Example 4 1% | 3.22E+06 | <1 | | 1.03E+05 | 2.3 | |
| | 1.67E+04 | | 2.9 | 0.00E+00 | | >5 |
| Example 5 1% | 1.34E+07 | <1 | | 1.54E+07 | <1 | |
| | 7.86E+06 | | <1 | 3.58E+06 | | <1 |
| Example 1 1% | 1.20E+04 | 3.1 | | 0.00E+00 | >5 | |
| | 0.00E+00 | | >5 | 0.00E+00 | | >5 |

In general, the activity is higher when ethanolic stock solutions are used instead of stock solutions in water (table (b)). However the compound is not stable over a longer period of time in ethanolic solution so that fast use is preferable.

TABLE (b)

| Test concentration | S. aureus | log reduction | | E. coli | log reduction | |
|---|---|---|---|---|---|---|
| 1% | cfu/mL | 5 min. | 30 min. | cfu/mL | 5 min. | 30 min. |
| Example 1 1% in EtOH | 3.45E+06 | 1 | | 1.37E+06 | 1.2 | |
| | 1.10E+04 | | 3.3 | 0.00E+00 | | >5 |
| Example 1 1% in H₂O | 6.98E+06 | <1 | | 4.62E+06 | <1 | |
| | 1.12E+06 | | 1.3 | 2.09E+06 | | 1.1 |
| Example 1 1% in H₂O | 8.19E+06 | <1 | | 5.03E+06 | <1 | |
| | 9.55E+05 | | 1.3 | 1.97E+06 | | 1.1 |

An anionic siloxane with phosophonic end group is tested in the same way and shows the following microbicidal activity (table (c)):

TABLE (c)

| | | log | | | log | |
|---|---|---|---|---|---|---|
| Test | | reduction | | | reduction | |
| concentration | S. aureus | 5 | 30 | E. coli | 5 | |
| 1000 ppm | cfu/mL | min. | min. | cfu/mL | min. | 30 min. |
| Inoculum | 3.80E+08 | | | 5.91E+08 | | |
| H₂O reference 5′ | 2.53E+07 | | | 2.13E+07 | | |
| H₂O reference 30′ | 2.50E+07 | | | 2.49E+07 | | |
| Example 2 | 1.42E+03 | 4.3 | | 1.50E+04 | 3.2 | |
| 1000 ppm | 0.00E+00 | | >5 | 0.00E+00 | | >5 |

### Example 7

### Fungicidal Use and Activity

Fungicidal activity is tested according to European Standard EN12175. A fungal spore suspension with a spore cell count of about 10⁶ cfu/ml is contacted with appropriate concentrations of the specific substances (Examples) mentioned in the tables below and the residual spore cell count is determined after incubation times of 30 and 60 min at room temperature under continuous stirring. Penicillium funiculosum (DSM 1960), Aspergillus niger (DSM 1957/ATCC 6275) and Aureobasidium pullulans (DSM 2404) are tested as important mold strains. DSM refers to microorganisms obtainable under the numbers given at the DSMZ (Deutsche Sammlung von Mikroorganismen and Zellkulturen GmbH, Braunschweig, Germany). ATCC numbers refer to organisms obtainable from the American Type Culture Collection (see Example 6). The results are given as a log reduction at each incubation time in comparison to a water control in table (d):

TABLE (d)

| | | | log | | | log | | | log | |
|---|---|---|---|---|---|---|---|---|---|---|
| Test conc. | | P. funic. | reduction | | A. niger | reduction | | A. pull. | reduction | |
| 1% | | cfu/mL | 30 min | 1 h | cfu/mL | 30 min | 1 h | cfu/mL | 30 min | 1 h |
| Inoculum | | 6.2E+07 | | | 3.0E+07 | | | 8.0E+06 | | |
| H2O | 30′ | 5.1E+06 | | | 1.5E+06 | | | 3.7E+05 | | |
| | 1 h | 4.6E+06 | | | 1.5E+06 | | | 3.2E+05 | | |
| 1% | 30′ | 4.8E+05 | 1.0 | | 1.5E+04 | 2.0 | | 1.3E+05 | <1 | |
| Example 4 | 1 h | 1.14E+05 | | 1.6 | 1.3E+04 | | 2.1 | 9.0E+04 | | <1 |
| 1% | 30′ | 2.5E+06 | <1 | | 7.0E+05 | <1 | | 3.5E+05 | <1 | |
| Example 5 | 1 h | 2.7E+06 | | <1 | 6.0E+05 | | <1 | 3.4E+05 | | <1 |
| 1% | 30′ | 4.0E+06 | <1 | | 1.1E+06 | <1 | | 2.1E+05 | <1 | |
| Example 1 | 1 h | 3.8E+06 | | <1 | 1.2E+06 | | <1 | 2.2E+05 | | <1 |

### Example 8

### Antiadhesive Use and Effects

The antiadhesive efficacy (inhibition of adhesion of microorganisms) is tested after temporarily attaching various compounds to PMMA test specimen by incubation of the test specimen in a 0.5% solution in ethanol. The coated specimen are then dried and afterwards incubated in a suspension of Staphylococcus aureus cells for 1 hour at 30° under shaking. After some washing steps, the attached cells are detected via binding of a primary antibody and—after incubation of night and again washing steps—binding of a secondary antibody with alkaline phosphatase attached. The adhesion is then determined in a colorimetric assay in comparison to an untreated PMMA test specimen, which is calculated as showing 100% adhesion. For results see table (e):

TABLE e

| | test | | |
|---|---|---|---|
| Test compound | concentration | solvent | results on PMMA |
| Example 4 | 0.50% | EtOH | 46% |
| Example 5 | 0.50% | EtOH | 50% |
| Example 1 | 0.50% | EtOH | 85% |

### Example 9

### Use and Efficacy on Polypropylene Non-Woven

A solution of Example 1 in a solvent (tetrahydrofurane and/or acetone) is prepared with a concentration of 1 or 2% of the compound of Example 1. This solution is sonicated for 1 min to ensure complete dissolution of the test acid compound. Then polypropylene non-woven (10×10 cm) is dipped into the solution (100 ml) and left under sonication for 1 min. The non-woven is taken out of the solution and dried on air at room temperature for some hours. This non-woven is then used for microbial testing as follows:

The efficacy of Example 1 is tested after application onto polypropylene non-woven material according to a modified AATCC-100 standard for assessing antibacterial activity on textiles. Overnight cultures of two test bacteria are contacted with the treated non-woven material by pipetting a diluted suspension onto the surface of the material. The non-woven is then incubated for 24 hours at 37° C., and then the cell count is determined by elution of the materials into a neutralizing medium, dilution series and plate count technique. As in the basic test for microbicidal activity (Example 6), Staphylococcus aureus is used as the gram+ and Escherichia coli as the gram-organism.

The results are given in tables (f) and (g) as cell count directly after inoculation and after 24 hours incubation time in comparison to an untreated polypropylene non-woven as the blank.

TABLE f

S. aureus ATCC 6538

| | 0 hours | 24 hours |
|---|---|---|
| Samples | [cfu/sample] | [cfu/sample] |
| Untreated nonwoven | 9.0 × 10⁴ | 7.8 × 10⁷ |
| | 7.6 × 10³ | 6.5 × 10⁷ |
| Example 1 (1%) treated nonwoven | | <100 |
| | | 6.9 × 10⁵ |
| Example 1 (2%) treated nonwoven | | <100 |
| | | <100 |

TABLE g

E. coli ATCC 10536

| | 0 hours | 24 hours |
|---|---|---|
| Samples | [cfu/sample] | [cfu/sample] |
| Untreated nonwoven | 1.6 × 10⁵ | 1.4 × 10⁸ |
| | 1.7 × 10⁵ | 1.1 × 10⁸ |
| Example 1 (1%) treated nonwoven | | 9.4 × 10⁶ |
| | | 2.8 × 10⁷ |
| Example 1 (2%) treated nonwoven | | 2.2 × 10³ |
| | | 920 |

The blanks show full growth of both organisms over the incubation time, the non-woven treated with 1% of Example 1 shows already some but inconsistent activity against S. aureus, which is probably due to the treatment procedure, and no activity against E. coli. When applied at 2%, the compound shows full activity against S. aureus and high activity against E. coli.

### Example 10

### Examples of Antibacterial Preparations (X=Preferred Combinations) of the Present Invention

### A. Personal Care Compositions

O/W systems:

| Ingredients | 1 | 2 | 3 | 4 | 5 | 6 | 7 | 8 |
|---|---|---|---|---|---|---|---|---|
| Emulsifiers | | | | | | | | |
| Potassium Cetyl Phosphate 2%-5% | X | | | | | | | |
| Cetearyl Alcohol/Dicetyl Phosphate/Ceteth-10 Phosphate | | X | | | | | | |
| 2%-6% | | | | | | | | |
| Sodium Stearyl Phtalamate 1%-2% | | | X | | | | | |
| Cetearyl Alcohol/Behentrimonium Methosulfate 1%-5% | | | | X | | | | |
| Quaternium-32 1%-5% | | | | | X | | | |
| Dimethicone copolyol/Caprylic/Capric Triglyceride (1%-4%) | | | | | | X | | |
| Steareth-2/Steareth-21 2%-5% | | | | | | | X | |
| Polyglyceryl Methyl Glucose Distearate 1%-4% | | | | | | | | X |
| Lipophilic emollient/dispersant oil 15%-20% | X | X | X | X | X | X | X | X |
| Fatty Alcohols and/or Waxes 1%-5% | X | X | X | X | X | X | X | X |
| Thickeners (water swellable thickeners) 0.5%-1.5% | X | X | X | X | X | X | X | X |
| Preservatives 0.5%-1% | X | X | X | X | X | X | X | X |
| Chelating agents (such as EDTA) 0%-0.2% | X | X | X | X | X | X | X | X |
| Antioxidants 0.05%-0.2% | X | X | X | X | X | X | X | X |
| Water deionized Qs 100% | X | X | X | X | X | X | X | X |
| Perfume oils 0.1%-0.4% | X | X | X | X | X | X | X | X |
| Antimicrobial agent of Example 1, 2, 3, 4, or 5: | X | X | X | X | X | X | X | X |
| 0.1%-20% | | | | | | | | |

Stick products

| Ingredients | 1 |
|---|---|
| Waxes 15%-30% | X |
| Natural and silicone oils 20%-75% | X |
| Lanoline derivatives 5%−>50% | X |
| Esters of lanolin | x |
| Acetylated lanolin | x |
| Lanolin oil | x |
| Colorants and pigments 10%-15% | X |
| Antioxidants 0.1%-0.8% | X |
| Perfume oils 0.1%-2% | X |
| Preservatives 0.1%-0.7% | X |
| Antimicrobial agent of Example 1, 2, 3, 4, or 5: | X |
| 0.1%-20% | |

Conditioning Shampoos

| Ingredients | 1 |
|---|---|
| Primary surfactants (listed previously) 5%-10% | X |
| Secondary surfactants (listed previously) 5%-15% | X |
| Foam Stabilizers (listed previously) 0%-5% | X |
| Water deionized 40%-70% | X |
| Actives 0-10% | X |
| Conditioners | x |
| Refatting agents | x |
| Moisturizing agents | x |
| Thickeners/Rheology mofifiers 0%-3% | X |
| Humectants 0%-2% | X |
| PH adjusting agents 0%-1% | X |
| Preservatives 0.05%-1% | X |
| Perfume oils 0.1%-1% | X |
| Antioxidants 0.05%-0.20% | X |
| Chelating Agents (EDTA) 0%-0.2% | X |
| Opascifying agents 0%-2% | X |
| Antimicrobial agent of Example 1, 2, 3, 4, or 5: | X |
| 0.1%-20% | |

### B. Home and Fabric Care Formulations

| | Formulation | | | | | | | | | | |
|---|---|---|---|---|---|---|---|---|---|---|---|
| Components | 1 | 2 | 3 | 4 | 5 | 6 | 7 | 8 | 9 | 10 | 11 |
| Antimicrobial agent of Example | 0.9 | 0.9 | 0.9 | 0.9 | 0.9 | 0.9 | 0.9 | 0.9 | 0.9 | 0.9 | 0.9 |
| 1, 2, 3, 4, or 5 | | | | | | | | | | | |
| dodecylbenzenesulfonic acid | 7.5 | | | | | | | 8.5 | | | |
| sodium | | 27 | 23.6 | 10 | 28 | | | | 20 | 24 | 6 |
| dodecylbenzenesulfonate | | | | | | | | | | | |
| sodium laureth sulfate 3 EO | 17 | 10 | | | | | | | | | |
| sodium lauryl sulfate | | | | | | 6 | | | | | 8 |
| coconut acid | 12.5 | | | 10 | 4 | 4 | 10 | | | 10 | |
| C₁₂₋₁₃ Pareth-7 | 10 | | | | | | | 26.9 | 27.8 | 25 | 4 |
| PEG-7 C₁₃ oxoalcohol | | | | 20 | 9 | 14.5 | 12 | 29 | 26 | | |
| PEG-8 C₁₃₋₁₅ fatty alcohol | | | | | | | 10 | | | | |
| alkyl polyglucoside | | | 5 | | | 1 | 2 | | | | |
| laureth-10 | | 5 | | | | | | | | | |
| PPG | | | | 2 | 3 | 8 | | | | | |
| sodium carbonate | | | 2 | | | | | | | | |
| sodium tripolyphosphate | | | 20 | | | | | | | | |
| potassium tripolyphosphate 50% | | 22 | | | | | | | | | |
| sodium cumenesulfonate 40% | | 25 | | | | | | | | | |
| trisodium citrate | 5.5 | | | | | 2 | | | | | 2 |
| lauryltrimonium chloride | 0.7 | | | | | | | | | | |
| polycarboxylate | | | | 13 | 18 | 15 | 10 | 23 | 16.2 | | |
| 2-propanol | 6 | | | 7 | 3 | | 4 | 9.5 | 8 | | |
| ethanol | 6 | | | | | | | | | | 9 |
| glycerol | | | | | | | | | | 20 | |
| propylene glycol | | | | | | | | | | 6 | |
| NaOH | 3.2 | | | 2 | 1 | 2.3 | 1.8 | 1.1 | | 1.8 | 4 |
| fluorescent whitening agent | 0.1 | 0.1 | 0.1 | 0.1 | 0.1 | 0.1 | 0.1 | | | | 0.1 |
| Tinopal CBS-x | | | | | | | | | | | |
| fluorescent whitening agent | | | | | | | | 0.1 | 0.1 | 0.1 | |
| Tinopal CBS-CL | | | | | | | | | | | |
| Soap | | | | | | | | | | | 7 |

1. A method of achieving an antimicrobial, preservative and/or microorganism adhesion inhibiting effect for the protection within or on the surface of an article and/or material which method comprises applying an acid compound, acid compound mixture or acid composition comprising,
an acid of the formula I, 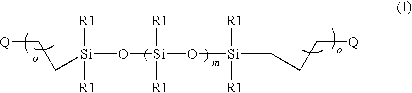
wherein R1=a linear or branched C₁-C₇-alky;
Q represents a linear or branched C₁-C₁₀-alkyl moiety substituted with 1-3 functional groups independently selected from the group consisting of COOH, P(O)(OH)₂ and S(O)₂(OH) which are attached via a C-atom to said moiety;
m represents an integer from 1 to about 20;
o represents an integer from 0 to 15; and
the acid preferably has a polydispersity of up to about 3 and a weight-average molecular weight of 600 to about 3000;
an acid of the general formula II, 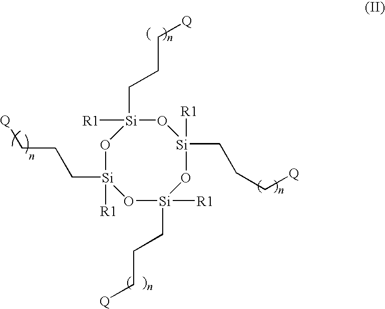
wherein
R1=methyl
Q represents a linear or branched C₁-C₁₀-alkyl moiety substituted with 1-3 functional groups independently selected from the group consisting of COOH, P(O)(OH)₂ and S(O)₂(OH) which are attached via a C-atom to said moiety; and
n represents an integer from 0 to 15;
and/or
an acid of the formula III, 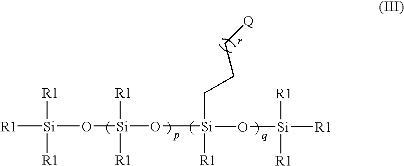
wherein:
R1=methyl
Q represents a linear or branched C₁-C₁₀-alkyl moiety substituted with 1-3 functional groups independently selected from the group consisting of COOH, P(O)(OH)₂ and S(O)₂(OH) which are attached via a C-atom to said moiety;
p represents an integer from 1 to about 20;
q represents an integer from 1 to about 20;
r represents an integer from 0 to 15; and
the acid preferably has a polydispersity of up to about 3 and a weight-average molecular weight of 600 to about 3000;
to an article and/or material in order to achieve an antimicrobial, preservative and/or microorganism adhesion inhibiting effect for the protection of said article and/or material;
with the proviso that, if the acid compound, acid compound mixture or acid composition used is one comprising only one or more acids of the formula III wherein Q is selected solely from the group consisting of linear or branched C₁-C₁₀-alkyl moieties substituted with two functional COON groups attached via a C-atom to said moiety, the method either is limited to a method for achieving an antimicrobial and/or a preservative effect, or, the method is limited to coating or impregnation of materials comprising synthetic polymers or polymer precursors or monomeric compounds, or of articles made from such materials or to the bulk addition to a material or an article; or in addition to one or more acids of the formula III at least another acid compound comprising an acid of the formula I as defined above, an acid of the formula II as defined above; and/or at least one acid compound comprising an additional different acid of the formula III as mentioned above wherein at least one moiety Q is present which is a linear or branched C₁-C₁₀-alkyl moiety substituted with 1 to 3 COOH, P(O)(OH)₂ and/or S(O)₂(OH) groups, with the proviso that in the case of a compound of the formula III said moiety Q does not carry only 2 COOH groups, is applied in addition;
and wherein an acid of the formula I, II and/or III the mentioned COOH, P(O)(OH)₂ and S(O)₂(OH) groups may also be partially or completely present in negatively charged form, so that the acids of the formula I, II and/or Ill may also be partially or completely be present as salts.

2. The method according to claim 1, wherein an acid compound, acid compound mixture or composition comprising at least one acid compound with an acid of the formula I is used.

3. The method according to claim 1, wherein an acid compound, acid compound mixture or composition comprising at least one acid compound with an acid of the formula II is used.

4. The method according to claim 1, wherein an acid compound, acid compound mixture or composition each comprising at least one acid compound with an acid of the formula III, and/or in each case a salt thereof is used wherein Q represents a linear or branched C₁-C₁₀alkyl moiety substituted with 1 to 3 functional groups independently selected from the group consisting of P(O)(OH)₂ and S(O)₂(OH).

5. The method according to claim 1, wherein an acid compound, acid compound mixture or composition comprising at least one acid compound with an acid of any one of the formulae I, II or III, and/or in each case a salt thereof, is used, wherein Q is a linear or branched C₁-C₁₀-alkyl moiety substituted with 1 to 3, P(O)(OH)₂ and S(O)₂(OH) groups.

6. The method according to claim 1, wherein as acid compound, acid compound mixture or composition one or more with an acid selected from the group consisting of those with the formulae given in the following table, and/or a salt thereof, is used:
Compound

| | |
|---|---|
| 1 | 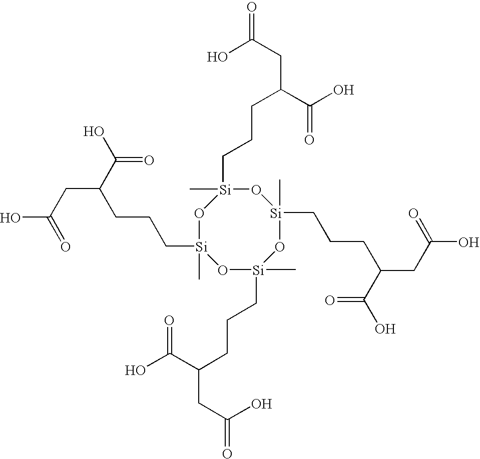 |
| 2 | 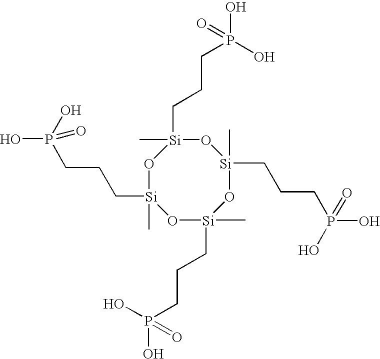 |
| 3 | 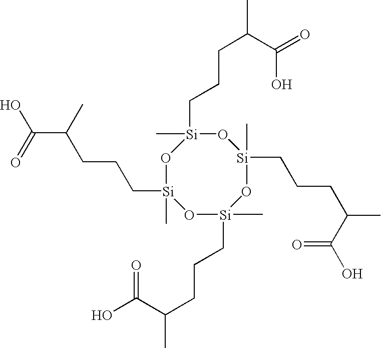 |
| 4 | 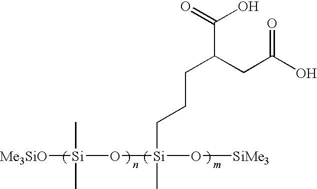 |
| 5 | 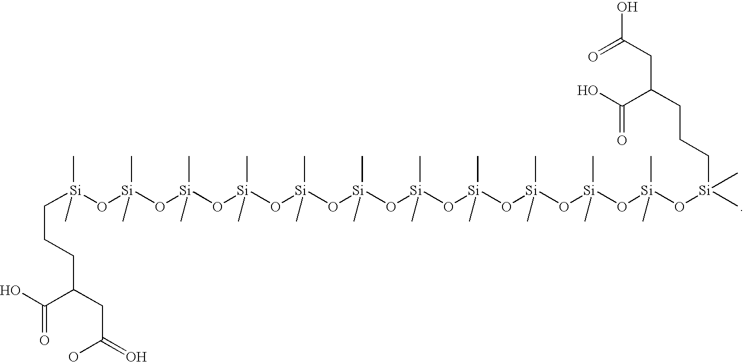 |

7. The method according to claim 1 to achieve an antimicrobial effect.

8. The method according to claim 1 which comprises applying a composition comprising one or more acid compounds of the formulae I, II or III and/or a salt thereof, wherein the acid compound(s) is present in 0.01 to 50% by weight.

9. The method according to claim 1 for the protection within or on the surface of an article and/or material wherein, the acid compound used is one comprising Q selected from the group consisting of linear or branched C₁-C₁₀-alkyl moieties substituted with COOH, and the materials comprise synthetic polymers or polymer precursors or monomeric compounds, and the articles are articles made from such materials.

10-11. (canceled)

12. An acid compound, compound mixture or composition comprising
at least one acid comprising at least one moiety Q which represents a linear or branched C₁-C₁₀-alkyl moiety substituted with 1-3 functional groups independently selected from the group consisting of COOH, P(O)(OH)₂ and S(O)₂(OH) which are attached via a C-atom to said moiety, wherein said at least one acid is selected from one or more acids from the group consisting of
one or more acids of the formula I, 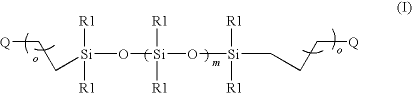
wherein R1=a linear or branched C₁-C₇-alkyl;
m represents an integer from 1 to about 20;
o represents an integer from 0 to 15; and
the acid preferably has a polydispersity of up to about 3 and a weight-average molecular weight of 600 to about 3000;
one or more acids of the general formula II, 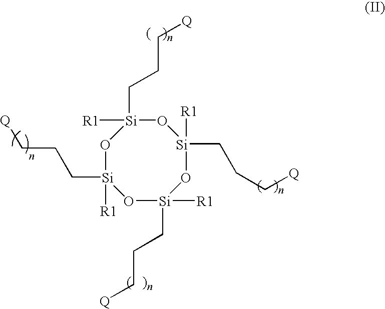
wherein
R1=methyl
n represents an integer from 0 to 15;
and/or
one or more acid of the formula III, 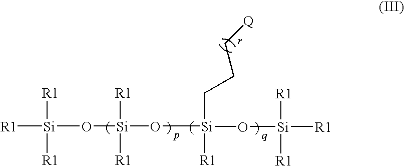
wherein:
R1=methyl
p represents an integer from 1 to about 20;
q represents an integer from 1 to about 20;
r represents an integer from 0 to 15; and
the acid preferably has a polydispersity of up to about 3 and a weight-average molecular weight of 600 to about 3000;
in free form and/or as salt(s);
with the proviso that if only acids of the formula III are present in a given compound, compound mixture or composition, at least in a part, preferably in all, of the molecules of the formula III one of the moieties Q is a linear or branched C₁-C₁₀-alkyl moiety substituted with 1 to 3 functional groups selected from P(O)(OH)₂ and S(O)₂(OH), or with 1 or 3 COOH groups.

13. An acid compound, compound mixture or composition according to claim 12, wherein the acid or acids are selected from those of the formula I and formula II.

14. An acid compound, compound mixture or composition according to claim 12, wherein the acid or acids are selected from those of the formula III wherein Q represents a linear or branched C₁-C₁₀-moeity substituted with 1 to 3 functional groups independently selected from the group consisting of P(O)(OH)₂ and S(O)₂(OH).

15. An acid compound, compound mixture or composition according to claim 12, wherein the acid or acids are selected from those wherein Q represents a linear or branched C₁-C₁₀-moeity substituted with 1 to 3 functional groups independently selected from the group consisting of P(O)(OH)₂ and S(O)₂(OH).

16. An acid compound, compound mixture or composition according to claim 12, comprising an acid, and/or a salt thereof, selected from the group of compounds given in the following table:
Compound

| | |
|---|---|
| 2 | 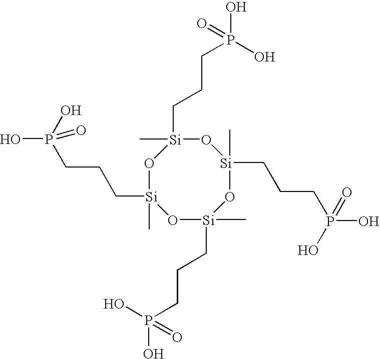 |
| 3 | 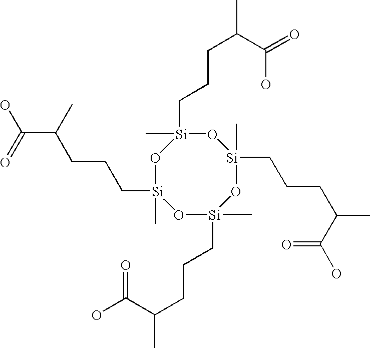 |
| 5 | 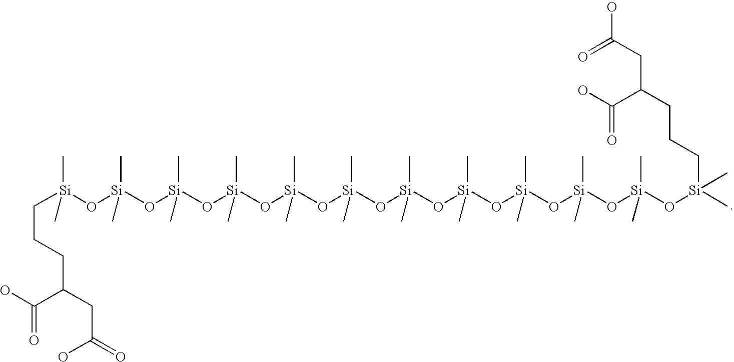 |

17. A process for the manufacture of an acid compound according to claim 12, comprising
(a) for the manufacture of an acid of the formula I, reacting a compound of the formula IA, 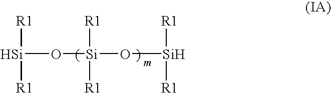
wherein R1 and m are as defined for an acid of the formula I,
under hydrosilylation conditions with a vinyl compound of the formula IV, 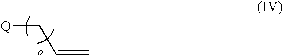
wherein Q and o are as defined for an acid of the formula I, or
(b) for the manufacture of an acid of the formula II, reacting a compound of the formula IIA, 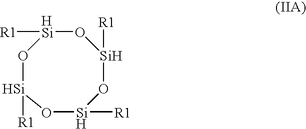
wherein R1 is as defined for an acid of the formula II,
under hydrosilylation conditions with a vinyl compound of the formula IV*, 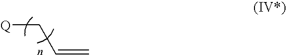
wherein Q and n are as defined for an acid of the formula II, or
(c) for the manufacture of an acid of the formula III, reacting a compound of the formula IIIA, 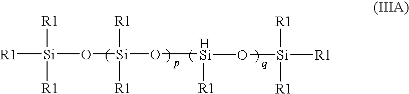
wherein R1, p and q are as defined for an acid of the formula III,
under hydrosilylation conditions with a vinyl compound of the formula IV**, 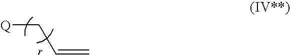
wherein Q and r as defined for an acid of the formula III,
with the proviso that in any of the starting materials of the formula IV, IV* and IV**, functional groups COOH, P(O)(OH)₂ and S(O)₂(OH) are present in protected form;
and subsequently removing the protection from the functional groups;
or leading two or more of the reactions under (a), (b) and (c) and the subsequent removal of protecting groups in parallel in order to directly obtain mixtures of acid compounds with acids of the formula I, II and/or III;
and, optionally conversion into a salt of an acid compound obtained according to a process according to a), b) and/or c) or conversion of one acid salt is a different salt with a different cation.

18. The method according to claim 2, wherein the acid compound, acid compound mixture or composition comprising at least one acid compound with an acid of the formula I is used in the absence of acids of the formula II and III.

19. The method according to claim 3, wherein the acid compound, acid compound mixture or composition comprising at least one acid compound with an acid of the formula II is used in the absence of acids of the formula I and Ill, and/or in each case a salt thereof.

20. The method according to claim 4, wherein the acid compound, acid compound mixture or composition each comprising at least one acid compound with an acid of the formula III, and/or in each case a salt thereof is used in the absence of acids of the formula I and II.

21. The method according to claim 8 which comprises applying a composition comprising one or more acid compounds of the formulae I, II or III and/or a salt thereof, wherein the acid compound(s) is present in 0.01 to 10% by weight.
